(19) United States
(12) Patent Application Publication
(10) Pub. No.: US 2025/0161282 A1
(43) Pub. Date: May 22, 2025

(54) PHARMACEUTICAL PYRIDYL COMPOSITIONS FOR TREATING AND PREVENTING DELAYED GRAFT FUNCTION

(71) Applicant: Unicycive Therapeutics, Inc.

(72) Inventors: Guru REDDY, Los Altos, CA (US); Satyanarayana Medicherla, Cupertino, CA (US); Shalabh Gupta, Mountain View, CA (US)

(21) Appl. No.: 18/947,766

(22) Filed: Nov. 14, 2024

Related U.S. Application Data
(60) Provisional application No. 63/599,665, filed on Nov. 16, 2023.
(60) Provisional application No. 63/561,059, filed on Mar. 4, 2024.

(51) Int. Cl. A61K 31/4425; A61K 31/4402; A61K 31/4545; A61P 13/12

(52) U.S. Cl. CPC A61K 31/4425; A61K 31/4402; A61K 31/4545; A61P 13/12

(57) ABSTRACT

Disclosed herein are pharmaceutical compositions in solution form and methods of treating diseases or conditions related to kidney transplant, particularly Delayed Graft Function. The composition and method of treatment and prevention includes administration of specific conjugated nicorandil compounds.

### CROSS-REFERENCE TO RELATED APPLICATION

This application claims priority to and the benefit of U.S. Provisional Application No. 63/599,665, filed Nov. 16, 2023, and U.S. Provisional Application No. 63/561,059, filed Mar. 4, 2024, which are incorporated by reference herein in their entirety for all purposes.

### FIELD

Described herein are compounds, pharmaceutical compositions and medicaments containing such compounds, and methods of using such compounds and compositions for treating diseases or conditions.

### BACKGROUND

Kidney diseases such as chronic kidney disease, acute kidney failure and acute kidney disease are major health concerns in USA. Approximately 20 million people in the United States are currently affected by chronic kidney disease (CKD), with half a million of these diagnosed with the most severe form of known as end-stage renal disease (ESRD).

In many instances, a kidney transplant is necessary. However, in as many as 50% of transplant cases, the immediate post-kidney transplant course is complicated by delayed graft function (DGF) and ischemia reperfusion (IR) injury. Ischemia reperfusion (IR) injury is inherent to renal graft function, wherein DGF is primarily a consequence of IR injury (IRI) resulting in post-ischemic acute tubular necrosis (ATN). IRI impairs graft function typically during the first week after transplantation.

IR injury is a complex phenomenon that induces tubular necrosis/injury through a bi-phasic process. Ischemia initiates the injury by deprivation of energy (via ATP depletion due to mitochondrial dysfunction) needed to maintain ionic gradients and homeostasis, which may ultimately lead to cellular dysfunction and tubular injury/death due to oxidative stress and apoptosis. Reperfusion exacerbates tubular damage triggering an inflammatory reaction in which oxidate stress and reactive oxygen species (ROS) participate.

Acute kidney injury occurs in kidney transplant patients and in about 30% of the patients it progresses to delayed graft function (DGF). The most accepted definition of DGF is the need for dialysis within the first week of transplantation. Two leading contributors of graft failure are acute rejection and acute kidney injury occurring during the transplant process. While acute rejection can be managed by immunosuppressive therapies, currently there are no FDA approved drugs for the prevention and/or treatment of DGF associated acute kidney injury (DGF-AKI).

Accordingly, there is a need for formulations and methods for treating and preventing a variety of diseases related to kidney transplant that are safe and effective compared to conventional treatments.

### SUMMARY

Without intending to be bound by any theory of operation, since increasing evidence indicates that mitochondrial dysfunction can be an important factor in the pathogenesis of AKI induced by IR injury, treatments aimed at protecting mitochondria should be effective in AKI.

The compounds and formulations disclosed herein can deliver nicorandil (used to treat angina), which binds to K_{ATP} channels and regulates mitochondrial permeability transition pore (MPTP), expressed on mitochondrial membranes of the renal tubular cells. Chronic opening of MPTP is a hallmark of mitochondrial dysfunction that can cause tubular injury/necrosis due to ATP depletion. The compounds and formulations provided herein may ameliorate DGF-AKI via closer of the MPTP and halting tubular injury.

Described herein are pyridyl compounds and pharmaceutical compositions thereof. Also described herein are methods for preventing, treating, and/or ameliorating diseases and conditions related to kidney transplant using the compounds and pharmaceutical compositions.

Also described herein are uses of the pyridyl compounds and the pharmaceutical compositions in the treatment of diseases or conditions related to kidney transplant. Further described are pharmaceutical compositions that include the pyridyl compounds.

In one aspect, provided herein are methods for preventing, treating, or ameliorating in a mammal in need thereof a disease or condition that is related to a kidney transplant, which comprises administering to the mammal an effective disease-treating or condition-treating amount of a compound according to Formula (I):

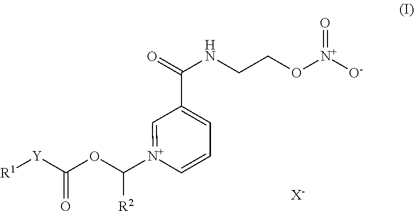

or a pharmaceutical composition thereof;
wherein X− is a counter ion; Y is —C(H)₂—, —O—, or —
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

In some embodiments, Y is —C(H)₂—. In some embodiments, Y is —O—. In some embodiments, Y is —N(R³)—. In particular embodiments, Y is —N(H)—.

In some embodiments, R² is H, Me, Et, or i-Pr. In a particular embodiment, R² is H.

In some embodiments, R¹ is ethyl substituted with cyclohexyl.

In some embodiments, R¹ is 2-cyclohexyl-ethyl.

In some embodiments, the compound is:

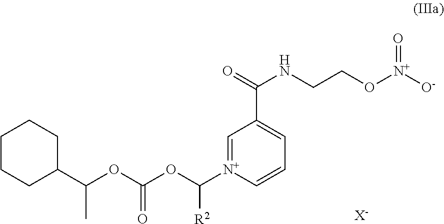

wherein X⁻, and R² are as described for formula (I) herein.

In some embodiments, the compound is:

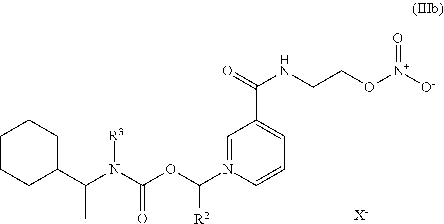

wherein X⁻, and R² are as described for formula (I) herein.

In some embodiments, with respect to formula (IIIa) or (IIIb), R² is H or Me. In some embodiments, R³ is H, Me, Et, or i-Pr. In particular embodiments, each R² and R³ is H.

In some embodiments, with respect to formula (IIIa) or (IIIb), the compound is an R-isomer. In some embodiments, with respect to formula (IIIa) or (IIIb), the compound is an S-isomer.

In some embodiments, the compound is:

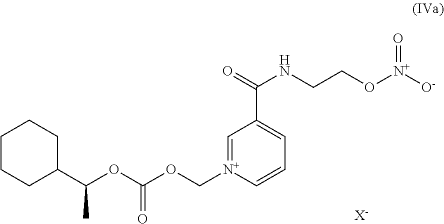

wherein X⁻ is as described for formula (I) herein.

In some embodiments, the compound is:

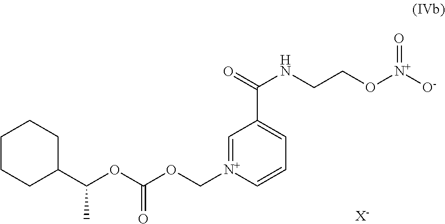

wherein X⁻ is as described for formula (I) herein.

In some embodiments, the compound is:

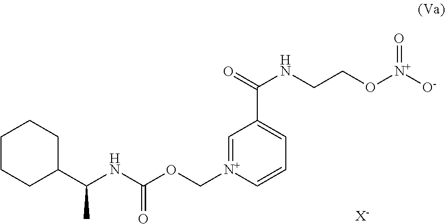

wherein X⁻ is as described for formula (I) herein.

In some embodiments, the compound is:

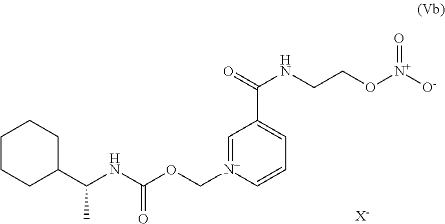

wherein X⁻ is as described for formula (I) herein.

In some embodiments, X⁻ includes one or two counter ions or more independently selected from iodide, chloride, fluoride, bromide, acetate, carbonate, chromate, citrate, fumarate, lactate, malonate, mesylate, nitrate, phosphate, tartrate, trifluoroacetate, succinate, sulphate, and sulphonate.

In some embodiments, X⁻ is Cl⁻. In another embodiment, X⁻ is Br⁻. In another embodiment, X⁻ is I⁻. In another embodiment, X⁻ is F⁻. In another embodiment, X⁻ is MeSO₂O⁻.

In one aspect, provided herein are methods for preventing, treating, or ameliorating in a mammal in need thereof a disease or condition that is related to a kidney transplant, which comprises administering to the mammal an effective disease-treating or condition-treating amount of nicorandil, or a derivative or prodrug thereof, or a pharmaceutically acceptable salt thereof.

In another aspect, the provided herein are pharmaceutical compositions comprising a therapeutically effective amount of a compound of formula (I) and a pharmaceutically acceptable excipient. In some embodiments, the pharmaceutical composition comprising the compound of formula (I) is formulated in a solution form for a route of administration selected from oral, oral mucosal, parenteral, nasal, or ophthalmic.

In another aspect, provided herein are compounds that are chemically stable and that can be synthesized by techniques known in the art, as well as those set forth herein.

Also provided are articles of manufacture including packaging material, an injectable pharmaceutical formulation provided herein, within the packaging material, and a label that indicates that the compound or composition, or pharmaceutically acceptable salt, pharmaceutically active metabolite, pharmaceutically acceptable prodrug, or pharmaceutically acceptable solvate thereof, is used for treating a disease or condition.

In any of the aforementioned embodiments are some embodiments in which administration is enteral, parenteral, or both, and wherein (a) an effective amount of a provided compound is systemically administered to the mammal; (b) an effective amount of a provided compound is intravenously administered to the mammal; or (c) an effective amount of a provided compound is administered by injection to the mammal.

In some embodiments, the present invention provides, methods for treating a disease or condition comprising administering to a patient in need the pharmaceutical composition of the present invention.

In some embodiments, the disease is selected from Transplant related Ischemia, Acute Kidney Injury (AKI) post kidney transplant, Delayed Graft Function (DGF), Primary Non Function (PNF), Prolonged Graft Ischemia, Primary Graft Failure, Rejection with Chronic Graft Failure, Ischemia Reperfusion (IR), Post-ischemic Acute Tubular Necrosis (ATN), Chronic Kidney Disease, Acute Kidney Failure, and/or Acute Kidney Disease and combinations thereof.

In a particular embodiment, the disease or condition is Delayed Graft Function.

In a particular embodiment, the disease or condition is Prolonged Graft Function.

In a particular embodiment, the disease or condition is Ischemia Reperfusion.

In a particular embodiment, the disease or condition is Post-ischemic Acute Tubular Necrosis.

In a particular embodiment, the disease or condition is Rejection with Chronic Graft Failure.

In a particular embodiment, the method comprises administering daily to the mammal an effective disease-treating or condition-treating amount of a compound according to formula (I) starting before the time of a kidney transplant.

In a particular embodiment, the method comprises administering daily to the mammal an effective disease-treating or condition-treating amount of a compound according to formula (I) starting at the time of a kidney transplant.

In a particular embodiment, the method comprises administering daily to the mammal an effective disease-treating or condition-treating amount of a compound according to formula (I) starting after the time of a kidney transplant.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### BRIEF DESCRIPTION OF DRAWINGS

FIG. 1 is a graph indicating the urinary β2-MG levels for various treatment groups.

FIGS. 2A-2C are graphs indicating the serum creatinine for a variety of treatment groups.

FIGS. 3A-3C are graphs indicating the urinary ACR for a variety of treatment groups.

FIG. 4 is a graph indicating BUN for various treatment groups.

FIGS. 5A-5B are graphs indicating Urinary NGAL for various treatment groups.

FIGS. 6A-6C are graphs indicating Proximal Tubular Injury for various treatment groups.

FIGS. 7A-7E are histology images for Proximal Tubular Injury for various treatment groups.

FIG. 8 is a graph indicating Urinary β2-MG levels for various treatment groups.

FIGS. 9A-9C are grafts indicating serum creatinine, BUN, and urinary NGAL for various treatment groups.

FIG. 10A provides nicorandil: serum creatinine, following administration of nicorandil, intraperitoneal route. FIG. 10B provides urinary ACR, following administration of nicorandil, intraperitoneal route. FIG. 10C provides tubular damage/injury, following administration of nicorandil, intraperitoneal route. FIG. 10D provides tubular damage/injury images, following administration of nicorandil, intraperitoneal route: Arrows point to healthy proximal tubules in No IR and IR+Nicorandil panels whereas in IR group arrows point towards lysed tubules.

### DETAILED DESCRIPTION

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications, or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In some embodiments, an alkyl comprises five to fifteen carbon atoms (e.g., C₅-C₁₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted as defined and described below and herein.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In some embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted as defined and described below and herein.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In some embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted as defined and described below and herein.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted as defined and described below and herein.

“Alkenylene” or “alkenylene chain” refers to a straight or branched divalent hydrocarbon chain linking the rest of the molecule to a radical group, consisting solely of carbon and hydrogen, containing at least one double bond, and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted as defined and described below and herein. “Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In some embodiments, a carbocyclyl comprises five to seven carbon atoms. The carbocyclyl is attached to the rest of the molecule by a single bond.

Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted as defined and described below and herein. “Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In some embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen, and sulfur. The radicals may be fused with an aryl or heteroaryl. Heterocycloalkyl rings can be formed by three to 14 ring atoms, such as three, four, five, six, seven, eight, nine, or more than nine atoms.

Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

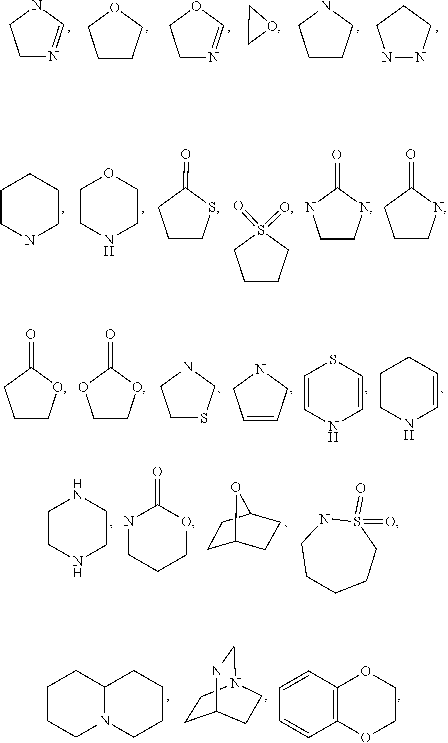

and the like. The term heteroalicyclic also includes all ring forms of the carbohydrates, including but not limited to the monosaccharides, the disaccharides, and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted as defined and described below and herein.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Imino” refers to the =NH radical.

“Thioxo” refers to the =S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus, or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule.

Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “trihalomethanesulfonyl” refers to a group of formula X₃CS(═O)₂— where Y is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls andheteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

As described herein, compounds provided herein may be “optionally substituted”. In general, the term “substituted,” whether preceded by the term “optionally” or not, means that one or more hydrogens of a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}2; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁-4 straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)O₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each RI is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “nucleophile” or “nucleophilic” refers to an electron rich compound, or moiety thereof.

The term “electrophile”, or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition, or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder, or condition by administration of a particular compound or pharmaceutical composition refers to any lessening of severity, delay in onset, slowing of progression, or shortening of duration, whether permanent or temporary, lasting, or transient that can be attributed to or associated with administration of the compound or composition.

“Bioavailability” refers to the percentage of the weight of compounds disclosed herein, such as, compounds of any of Formula (I)—(Vb) dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of Formula (I)—(Vb) are absorbed into the general circulation when the pharmaceutical composition is taken orally as compared to intravenous injection.

“Blood plasma concentration” refers to the concentration of compounds disclosed herein, such as, compounds of any of Formula (I)—(Vb) in the plasma component of blood of a subject. It is understood that the plasma concentration of compounds of any of Formula (I)—(Vb) may vary significantly between subjects, due to variability with respect to metabolism and/or possible interactions with other therapeutic agents. In accordance with some embodiments disclosed herein, the blood plasma concentration of the compounds of any of Formula (I)—(Vb) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of any of Formula (I)—(Vb) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (I)—(Vb), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder, or condition. An “enhancing-effective amount,” as used herein, refers to an amount adequate to enhance the effect of a therapeutic agent in the treatment of a disease, disorder, or condition. When used in a patient, amounts effective for this use will depend on the severity and course of the disease, disorder or condition, previous therapy, the patient's health status and response to the drugs, and the judgment of the treating physician.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, “preventing” the symptoms of a particular disease, disorder, or condition by administration of a particular compound or pharmaceutical composition refers to any lessening of the likelihood of the disease, disorder, or condition, or one or more symptoms thereof. In certain embodiments, this indicates a reduction in severity of the disease, disorder, or condition, or one or more symptoms thereof. In certain embodiments, this indicates a delay in the onset of the disease, disorder, or condition, or one or more symptoms thereof. In certain embodiments, this indicates a slowing of progression of the disease, disorder, or condition, or one or more symptoms thereof. In certain embodiments, this indicates a slowing of progression of the disease, disorder, or condition, or one or more symptoms thereof. In particular embodiments, prevention does not require complete elimination of the disease, disorder, or condition, or complete elimination of any one or more symptoms thereof.

As used herein, the IC₅₀ refers to an amount, concentration or dosage of a particular test compound that achieves a 50% inhibition of a maximal response, in an assay that measures such response.

As used herein, EC₅₀ refers to a dosage, concentration or amount of a particular test compound that elicits a dose-dependent response at 50% of maximal expression of a particular response that is induced, provoked, or potentiated by the particular test compound.

Methods described herein include administering to a subject in need a composition containing a therapeutically effective amount of one or more compounds described herein.

A number of animal models of are useful for establishing a range of therapeutically effective doses of pyridyl compounds for treating any of the foregoing diseases.

The therapeutic efficacy of a provided compound for one of the foregoing diseases can be optimized during a course of treatment.

### Compounds

In the following description of pyridyl compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

Described herein are compounds of any of Formulae (I)—(Vb). Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided. In some embodiments, when compounds disclosed herein contain an oxidizable nitrogen atom, the nitrogen atom can be converted to an N-oxide by methods well known in the art. In certain embodiments, isomers and chemically protected forms of compounds having a structure represented by any of Formula (I)—(Vb) are also provided.

In some embodiments, provided herein are pyridyl according to compounds of formula (I).

In some embodiments, provided herein is a compound according to formula (I) having the structure:

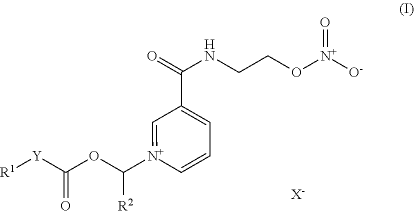

or a pharmaceutical composition thereof;
wherein X⁻ is a counter ion; Y is —C(H)₂—, —O—, or —N(R³)—;
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

In some embodiments, provided herein are uses of a compound according to formula (I):

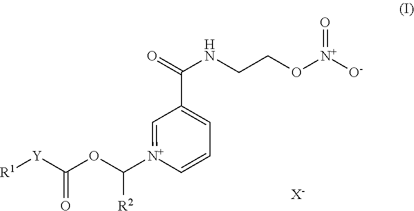

in a treatment of a disease or a condition related to Delayed Graft Function (DGF) in kidney transplant patients;
wherein X⁻ is a counter ion; Y is —C(H)₂—, —O—, or —N(R³)—;
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

In some embodiments, R² is H, Me, Et, or i-Pr.

In some embodiments, R² is H.

In some embodiments, Y is —C(H)₂—.

In some embodiments, Y is —O—.

In some embodiments, Y is —N(R³)—.

In some embodiments, the compound is according to formula (IIa), (IIb), or (IIc):

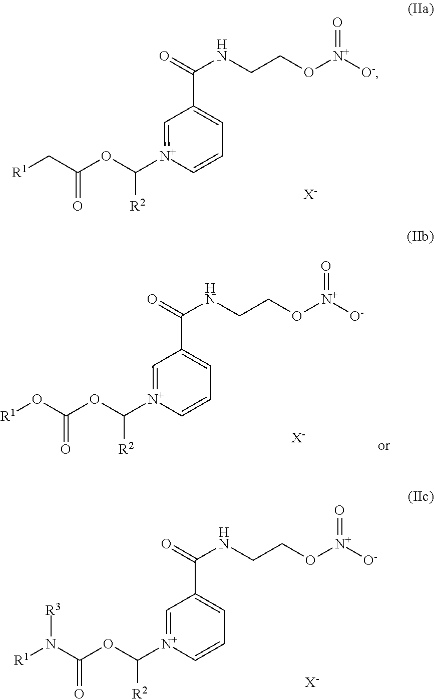

wherein
X⁻ is a counter ion;
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

In some embodiments, R¹ is substituted or unsubstituted C₁-C₄ alkyl.

In some embodiments, R¹ is C₁-C₄ alkyl, unsubstituted or substituted with C₃-C₇ cycloalkyl.

In some embodiments, R¹ is Me, Et, i-Pr, n-Pr, n-Bu, i-Bu, sec-Bu, or t-Bu.

In some embodiments, R¹ is Me or Et substituted with cyclopropyl, cyclobutyl, cyclopentyl or cyclohexyl.

In some embodiments, R¹ is C₃-C₇-cycloalkyl unsubstituted or substituted with Me or Et.

In some embodiments, R¹ is cyclopropyl, cyclobutyl, cyclopentyl or cyclohexyl.

In some embodiments, R³ is H or substituted or unsubstituted C₁-C₄ alkyl.

In some embodiments, R³ is H.

In some embodiments, R³ is Me, Et, i-Pr, n-Pr, n-Bu, i-Bu, sec-Bu, or t-Bu.

In some embodiments, R¹ and R³ are joined together to form a 4-7 membered substituted or unsubstituted heterocycloalkyl.

In some embodiments, R¹ and R³ are joined together to form pyrrolidinyl, piperidinyl, or morpholinyl.

In some embodiments, the compound is according to formula (IIIa), or (IIIb):

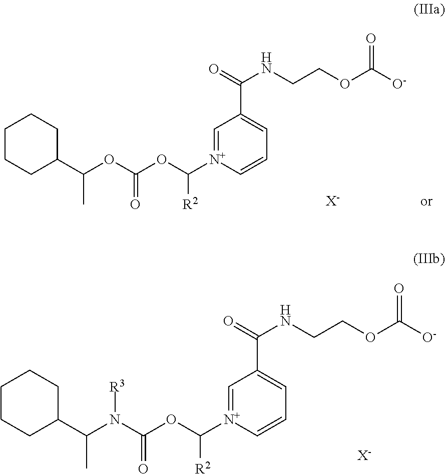

wherein
X⁻ is a counter ion;
R² is H or C₁-C₄ alkyl; and
R³ is H or substituted or unsubstituted C₁-C₄ alkyl.

In some embodiments, R³ is H, Me, Et, or i-Pr.

In some embodiments, R³ is H.

In some embodiments, R² is H or Me.

In some particular embodiments, the compound is according to formula (IIIb); and each R² and R³ is H.

In some embodiments, the compound is according to formula (IVa), or (IVb):

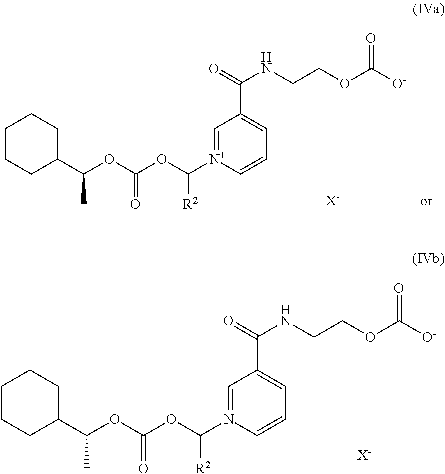

wherein X⁻ is a counter ion.

In some embodiments, the compound is according to formula (Va), or (Vb):

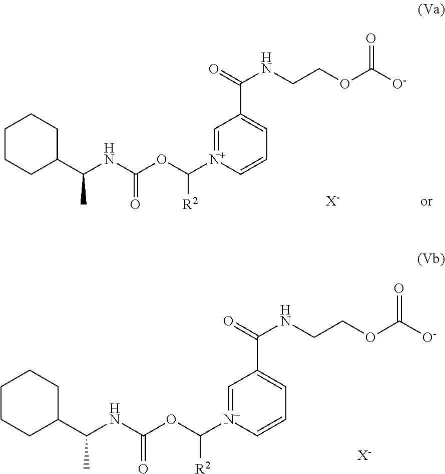

wherein X⁻ is a counter ion.

In some embodiments, the counter ion is any pharmaceutically acceptable anion. In some embodiment, the counter ion is a halide or a sulfonate. In a particular embodiment, the counter ion is F⁻, Cl⁻, Br⁻, I⁻. In another particular embodiment, the counter ion is benzene sulfonate or methyl sulfonate.

In some embodiments, X⁻ includes one or two counter ions independently selected from iodide, chloride, fluoride, bromide, acetate, carbonate, chromate, citrate, fumarate, lactate, malonate, mesylate, nitrate, phosphate, tartrate, trifluoroacetate, succinate, sulphate, and sulphonate.

In some embodiments, X⁻ is F⁻, Cl⁻, Br⁻, I⁻, or MeSO₂O⁻.

In a particular embodiment, X⁻ is Cl⁻, or I⁻. In a more particular embodiment, X⁻ is I⁻.

In some embodiments, the compound is nicorandil, or a derivative or prodrug thereof, or a pharmaceutically acceptable salt thereof. In some embodiments, the compound is nicorandil, or a pharmaceutically acceptable salt thereof. The derivative of nicorandil can be any derivative deemed suitable by the person of skill. The prodrug of nicorandil can be any prodrug deemed suitable by the person of skill. Exemplary derivatives and prodrugs of nicorandil are described, for example, in international patent publication WO 2012/137225 or WO 2020/190890, the contents of which are hereby incorporated by reference in their entireties.

In some embodiments, provided herein is a pharmaceutical composition comprising a compound according to formula (I).

In some embodiments, provided herein is a pharmaceutical composition comprising a therapeutically effective amount of a compound of formula (I), and a pharmaceutically acceptable excipient.

In some embodiments, the pharmaceutical composition is formulated in solution form for a route of administration selected from oral, oral mucosal, ophthalmic, nasal to parenteral administration.

In some embodiments, the compound is according to formula (Va).

In some embodiments, the compound is according to formula (Vb).

In some embodiments, the compound is nicorandil. In some embodiments, the compound is a derivative or prodrug of nicorandil.

In some embodiments, provided herein is a pharmaceutical composition comprising a pharmaceutically acceptable carrier and a pharmaceutically effective amount of a compound according to any one of the formulas described herein. In some embodiments, the compound is according to any one of Formula (I)—(Vb).

In some embodiments, the pharmaceutical composition is formulated in solution form for a route of administration selected from oral oral mucosal, parenteral, nasal, and ophthalmic administration.

In some embodiments, the carrier is a parenteral carrier.

In some embodiments, the carrier is an oral carrier.

In some embodiments, the carrier is an ophthalmic or nasal carrier.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

Throughout the specification, groups and substituents thereof can be chosen by one skilled in the field to provide stable moieties and compounds.

### Preparation of Compounds

Compounds of any of Formula (I)—(Vb) may be synthesized using synthetic reactions known to those of skill in the art or using methods known in the art. The reactions can be employed in a linear sequence to provide the compounds or they may be used to synthesize fragments which are subsequently joined by the methods known in the art.

The starting material used for the synthesis of the compounds described herein may be synthesized or can be obtained from commercial sources, such as, but not limited to, Aldrich Chemical Co. (Milwaukee, Wisconsin), Bachem (Torrance, California), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Additional methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography, and the like. Such materials may be characterized using conventional means, including physical constants and spectral data.

Compounds described herein may be prepared as a single isomer or a mixture of isomers.

In some embodiments, representative compounds of Formula (I) are prepared according to synthetic schemes and methods described in a US patent to Dugar et al., U.S. Pat. No. 9,359,376.

### Further Forms of Compounds

Compounds disclosed herein have a structure of Formula (I)—(Vb). It is understood that when reference is made to compounds described herein, it is meant to include compounds of any of Formula (I), (IIa)-(IIc), (IIIa)-(IIIb), (IVa)-(IVb), or (Va)-(Vb) as well as to all of the specific compounds that fall within the scope of these generic formulae, unless otherwise indicated.

Compounds described herein may possess one or more stereocenters and each center may exist in the R or S configuration. Compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diastereomeric mixtures can be separated into their individual diastereomers on the basis of their physical chemical differences by methods known, for example, by chromatography and/or fractional crystallization. In some embodiments, enantiomers can be separated by chiral chromatographic columns. In some embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

Methods and formulations described herein include the use of N-oxides, crystalline forms, or pharmaceutically acceptable salts of compounds described herein, as well as active metabolites of these compounds having the same type of activity. In some situations, compounds may exist as tautomers. All tautomers are included within the scope of the compounds presented herein. In addition, compounds described herein can exist in unsolvated as well as solvated forms with pharmaceutically acceptable solvents such as water, ethanol, and the like. Solvated forms of compounds presented herein are also considered to be disclosed herein.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine, and chlorine, such as ²H, ³H, ¹³C, ¹⁴C, ¹⁵N, ¹⁸O, ¹⁷O ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

The salts are recovered by using at least one of the following techniques: filtration, precipitation with a non-solvent followed by filtration, evaporation of the solvent, or, in the case of aqueous solutions, lyophilization.

Throughout the specification, groups and substituents thereof can be chosen by one skilled in the field to provide stable moieties and compounds.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition in solution form, as used herein, refers to a mixture of a compound described herein, such as, for example, compounds of any of Formula (I)—(Vb) with other chemical components, such as solvents, co-solvents, solubilizers, stabilizers, diluents, buffering or pH adjusting agents, preservatives, thickening agents, toxicity adjusting agents, antioxidants, and/or excipients used similarly for preparing pharmaceutical solutions by a person skilled in the art. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, and ammonium chloride. Such acids, bases and buffers are included in an amount required to maintain pH of the composition in an acceptable range.

In some embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The compositions described herein can be formulated for administration to a subject via any conventional means including, but not limited to, oral, parenteral (e.g., intravenous, subcutaneous, or intramuscular), buccal, intranasal, rectal, or transdermal administration routes. As used herein, the term “subject” is used to mean an animal, preferably a mammal, including a human or non-human. The terms patient and subject may be used interchangeably.

Pharmaceutical compositions including a compound described herein may be manufactured in a conventional manner, such as, by way of example only, by means of conventional mixing, dissolving, emulsifying, or other suitable processes.

Moreover, the pharmaceutical compositions described herein, which include a compound of any of Formula (I)—(Vb) can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition is in the form of a powder. In some embodiments, the pharmaceutical composition is in the form of a tablet, including but not limited to, a fast-melt tablet. Additionally, pharmaceutical compositions described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of any of Formula (I)—(Vb) with one or more pharmaceutical excipients to form a bulk blend composition. When referring to these bulk blend compositions as homogeneous, it is meant that the particles of the compound of any of Formula (I)—(Vb) are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical compositions will include at least one compound described herein, such as, for example, a compound of any of (I)—(Vb) as an active ingredient in free-acid or free-base form, or in a pharmaceutically acceptable salt form. In addition, the methods and pharmaceutical compositions described herein include the use of N-oxides, crystalline forms, as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

Formulations that include a compound of any of Formula (I)—(Vb) may include physiologically acceptable sterile aqueous or non-aqueous solutions, and sterile powders for reconstitution into sterile injectable solutions. Examples of suitable aqueous and non-aqueous carriers, diluents, solvents, or vehicles include water, ethanol, polyols (propylene glycol, polyethylene-glycol, glycerol, cremophor and the like), and suitable mixtures thereof. In an example, the injection formulation includes a non-water based solvent. In an example, the non-water based solvent includes ethanol, propylene glycol, polyethylene glycol, polysorbate 80 (e.g., Tween-80), Kolliphor (EL), or combinations thereof. Prevention of the growth of microorganisms can be ensured by various antibacterial and antifungal agents, such as parabens, chlorobutanol, phenol, sorbic acid, and the like. It may also be desirable to include isotonic agents, such as dextrose, sodium chloride, and the like.

For intravenous injections, compounds described herein may be formulated in aqueous solutions, preferably in physiologically compatible buffers such as Hank's solution, Ringer's solution, or physiological saline buffer. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. Aqueous injection suspensions may contain substances which increase the viscosity of the suspension, such as sodium carboxymethyl cellulose, sorbitol, or dextran. Alternatively, the active ingredient may be in powder form for constitution with a suitable vehicle, e.g., sterile pyrogen-free water, before use.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) glycerol, (b) methionine, (c) monothioglycerol, (d) EDTA, (e) ascorbic acid, (f) polysorbate 80, (g) polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

A “carrier” or “carrier materials” include any commonly used excipients in pharmaceutics and should be selected on the basis of compatibility with compounds disclosed herein, such as, compounds of any of Formula (I)—(Vb). Exemplary carrier materials include surfactants, solubilizers, stabilizers, lubricants, wetting agents, diluents, and the like. “Pharmaceutically compatible carrier materials” may include, but are not limited to, sodium chloride, carboxymethylcellulose sodium, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Viscosity modulating agents” include materials that control the diffusion and homogeneity of a drug through liquid media. Exemplary diffusion facilitators include, e.g., hydrophilic polymers, electrolytes, Tween® 60 or 80, PEG, polyvinylpyrrolidone (PVP; commercially known as Plasdone®), and the carbohydrate-based dispersing agents such as, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxypropylcellulose, hydroxypropylmethylcellulose phthalate, triethanolamine, polyvinyl alcohol (PVA), poloxamers (e.g., Pluronics F68®, F88®, and F108®, which are block copolymers of ethylene oxide and propylene oxide); and poloxamines (e.g., Tetronic 908©, also known as Poloxamine 908©), polyvinylpyrrolidone (povidone) K12, polyvinylpyrrolidone K17, polyvinylpyrrolidone K25, or polyvinylpyrrolidone K30, polyvinylpyrrolidone/vinyl acetate copolymer (S-630), polyethylene glycol having a molecular weight of about 300 to about 6000, or about 3350 to about 4000, or about 7000 to about 5400, sodium alginate, gums, e.g., gum tragacanth and gum acacia, guar gum, xanthan, including xanthan gum, sugars, polyethoxylated sorbitan monolaurate, polyethoxylated sorbitan monolaurate, carbomers, alginates, chitosans and combinations thereof.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution, normal saline, or combinations thereof. Diluents may also include mannitol, sorbitol, dextrose, and sodium chloride.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

A “measurable serum concentration” or “measurable plasma concentration” describes the blood serum or blood plasma concentration, typically measured in mg, g, or ng of therapeutic agent per ml, dl, or 1 of blood serum, absorbed into the bloodstream after administration. As used herein, measurable plasma concentrations are typically measured in ng/ml or g/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Stabilizers” include compounds such as any antioxidation agents, buffers, acids, preservatives, and the like.

“Steady state,” as used herein, is when the amount of drug administered is equal to the amount of drug eliminated within one dosing interval resulting in a plateau or constant plasma drug exposure.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In an example, the surfactant includes a nonionic surfactant, e.g., Kolliphor® EL (BASF). In some embodiments, surfactants may be included to enhance physical stability or for other purposes.

“Viscosity enhancing agents” include, e.g., methyl cellulose, xanthan gum, carboxymethyl cellulose sodium, hydroxypropyl cellulose, hydroxypropylmethyl cellulose, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated in solution form for administration to a subject via oral, oral mucosal, nasal, ophthalmic, and parenteral (e.g., intravenous, subcutaneous, or intramuscular). As used herein, the term “subject” is used to mean an animal, preferably a mammal, including a human or non-human. The terms patient and subject may be used interchangeably.

Pharmaceutical preparations can be obtained by mixing one or more excipient with one or more of the compounds described herein. Suitable excipients include, for example, fillers such as dextrose including sucrose, mannitol, glycerol, or sorbitol; thickening agents such as cellulose preparation, e.g., methylcellulose, hydroxypropylmethylcellulose, sodium carboxymethylcellulose; sodium alginate, or others such as polyvinylpyrrolidone (PVP or povidone).

The pharmaceutical dosage forms described herein can include a compound described herein and one or more pharmaceutically acceptable additives such as a compatible carrier, solvent, co-solvent, bulking agent, pH adjusting agent, surfactant, lubricant, colorant, sweetening & flavoring agent, diluent, solubilizer, stabilizer, penetration enhancer, wetting agent, anti-foaming agent, antioxidant, preservative, chelating agent, tonicity adjusting agent or one or more combination thereof.

Suitable antioxidants for use in the dosage forms described herein include, for example, e.g., butylated hydroxytoluene (BHT), sodium ascorbate, and tocopherol.

The above-listed additives should be taken as merely exemplary, and not limiting, of the types of additives that can be included in forms described herein. The amounts of such additives can be readily determined by one skilled in the art, according to the particular properties desired.

Suitable preservatives for the injectable formulations described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the injectable formulations described herein include, but are not limited to, methyl cellulose, xanthan gum, carboxymethyl cellulose, hydroxypropyl cellulose sodium, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

In addition to the additives listed above, the injectable formulations can also include inert diluents commonly used in the art, such as water or other solvents, solubilizing agents, polyethylene glycols, fatty acid esters of sorbitan, or mixtures of these substances, and the like.

### Examples of Methods of Dosing and Treatment Regimens

The compounds described herein can be used in the preparation of medicaments for the treatment of diseases or conditions. In addition, a method for treating any of the diseases or conditions described herein in a subject in need of such treatment, involves administration of pharmaceutical compositions containing at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, in a single dose ranging from 80 mg to 160 mg, 90 mg to 160 mg, 100 mg to 160 mg, 110 mg to 160 mg, 120 mg to 160 mg, 130 mg to 160 mg, 140 mg to 160 mg, or 150 mg to 160 mg. In an example, the single dose can be 80 mg, 90 mg, 100 mg, 110 mg, 120 mg, 130 mg, 140 mg, 150 mg, or 160 mg.

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, in multiple doses (e.g., BID). In an example, the dose can be in a range of 10 mg to 80 mg BID, 20 mg to 80 mg BID, 30 mg to 80 mg BID, 40 mg to 80 mg BID, 50 mg to 80 mg BID, 60 mg to 80 mg BID, or 70 mg to 80 mg BID. In an example, the dose can be 10 mg BID, 20 mg BID, 30 mg BID, 40 mg BID, 50 mg BID, 60 mg BID, 70 mg BID, or 80 mg BID. The multiple doses can be administered the days prior to the transplantation and extending 2 days, 3 days, 5 days, or 10 days post-transplantation.

In an example, the pharmaceutically acceptable salt can be selected from iodide, chloride, fluoride, bromide, acetate, carbonate, chromate, citrate, fumarate, lactate, malonate, mesylate, nitrate, phosphate, tartrate, trifluoroacetate, succinate, sulphate, and sulphonate, and combinations thereof. In some embodiments, the counter ion of Formula (I)—(Vb) is a pharmaceutically acceptable anion, wherein the counter ion can be a halide (e.g., F—, Cl—, Br—, I—) or a sulfonate (e.g., benzene sulfonate or methyl sulfonate).

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, wherein the administration is performed before the time of the kidney transplant. For example, before the time of the kidney transplant can be at least 24 hours, at least 12 hours, at least 6 hours, at least 3 hours, at least 1 hour, at least 30 minutes, or at least 15 minutes before the kidney transplant begins.

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, wherein the administration is performed at the time of the kidney transplant. In certain embodiments, “at the time of” indicates within 15 minutes, within 10 minutes, or within 5 minutes of transplant.

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, wherein the administration is performed immediately after the time of the kidney transplant. For example, after the time of the kidney transplant can be at least 24 hours, at least 12 hours, at least 6 hours, at least 3 hours, at least 1 hour, at least 30 minutes, or at least 15 minutes, after the kidney transplant is complete.

In an example, the methods described herein include administering at least one compound of any of Formula (I)—(Vb), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject, wherein the administration is performed at least once daily for at least 30 days. In an example, the first does can be administered the day of the transplant.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In some embodiments, particularly those related to graft or transplant function, the compound is administered before, during, or after the graft or transplant. In some embodiments, the compound is administered before graft or transplant. In some embodiments, the compound is administered 14 days, 10 days, 9 days, 8 days, 7 days, 6 days, 5 days, 4 days, 3 days, 2 days, 1 day, 12 hours, or 6 hours before graft or transplant. In some embodiments, the compound is administered after graft or transplant. In some embodiments, the compound is administered 14 days, 30 days, 15 days, 10 days, 9 days, 8 days, 7 days, 6 days, 5 days, 4 days, 3 days, 2 days, 1 day, 12 hours, or 6 hours after graft or transplant. In some embodiments, the compound is administered one day prior to transplantation and subsequently the following 2 days, 3 days, 4 days, and/or 5 days post transplantation. Following the initial administration, the compound can be administered on a schedule deemed suitable by the practitioner of skill. In certain embodiments, the compound is administered daily after the initial administration. In particularly embodiments, administration continues until a stopping point is reached according to the judgment of the practitioner of skill. In certain embodiments, the administration continues until graft or transplant function is satisfactory.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged powders in vials or ampoules. Aqueous suspension compositions can be packaged in single-dose non-reclosable containers. Alternatively, multiple-dose reclosable containers, vials, ampoules, or other routinely used container closure systems can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by using the present compounds.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded, or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

The examples below as well as throughout the application, the following abbreviations have the following meanings. If not defined, the terms have their generally accepted meanings.
aq=aqueous
Boc=tert-butyloxycarbonyl
t-BuOH=tertiary butanol
DCE=1,2-dichloroethane
DCM=dichloromethane
DIAD=diisopropyl azodicarboxylate
DIEA or DIPEA=N,N-diisopropylethylamine
DMAP=dimethylaminopyridine
DMF=dimethylformamide
DMSO=dimethylsulfoxide
ESI=electron spray ionization
EA=ethyl acetate
g=gram
HCl=hydrogen chloride
HPLC=high performance liquid chromatography
hr=hour
¹H NMR=proton nuclear magnetic resonance
IPA=isopropyl alcohol
KOAc=potassium acetate
LC-MS=liquid chromatography mass spectroscopy
M=molar
MeCN=acetonitrile
MeOH=methanol
mg=milligram
min=minute
ml=milliliter
mM=millimolar
mmol=millimole
m.p.=melting point
MS=mass spectrometry
m/z=mass-to-charge ratio
N=normal
NIS=N-iodosuccinimide
nM=nanomolar
nm=nanometer
Pd(dppf)Cl₂=[1,1′-Bis(diphenylphosphino)ferrocene]dichloropalladium(II)
PE=petroleum ether
PyBOP=benzotriazol-1-yl-oxytripyrrolidinophosphonium hexafluorophosphate
quant.=quantitative
RP=reverse phase
RT, rt or r.t.=room temperature
Sat.=saturated
TEA=triethylamine
TFA=trifluoroacetic acid
μL=microliter
μM=micromolar

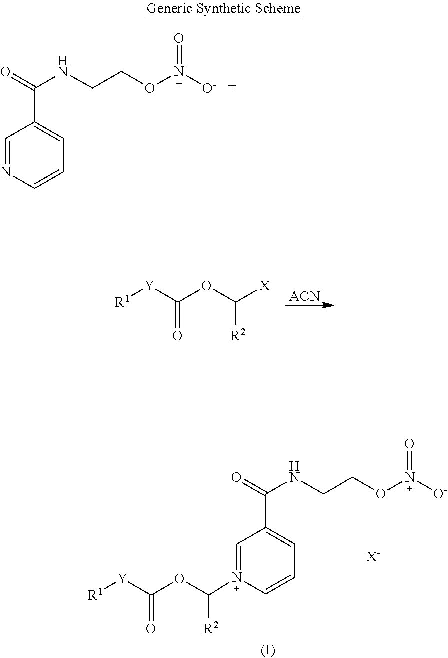

wherein X, Y, R¹, and R² are as described herein.

### Example 1

Compounds are prepared consistent with the procedures of international application nos. WO 2012/137225, filed Apr. 9, 2012, and WO 2020/190890, filed Mar. 16, 2020, the contents of which are hereby incorporated by reference in their entireties.

TABLE 1
Representative Compounds

| Compound # | Structure | Salt |
|---|---|---|
| Nicorandil | 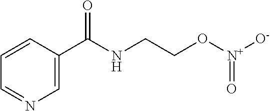 [SMILES: O=C(NCCO[N+](=O)[O-])c1cccnc1] | N/A |
| | C₈H₉N₃O₄ | |
| 1 | 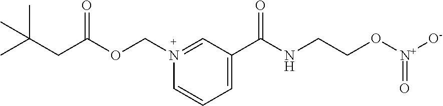 | Iodide |
| | C₁₅H₂₂N₃O₆⁺ | |
| 2 | 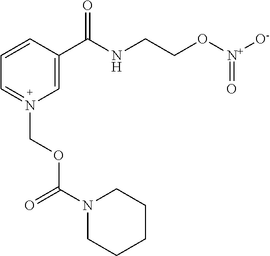 | Iodide |
| | C₁₅H₂₁N₄O₆⁺ | |
| 3 | 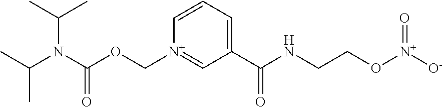 [SMILES: CC(C)N(C(=O)OC[n+]1cccc(C(=O)NCCO[N+](=O)[O-])c1)C(C)C] | Iodide |
| | C₁₆H₂₅N₄O₆⁺ | |
| 4 | 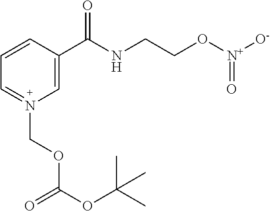 | Iodide |
| | C₁₄H₂₀N₃O₇⁺ | |
| 5 | 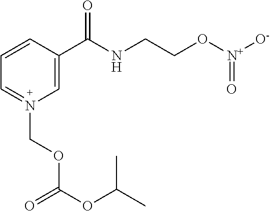 | Iodide |
| | C₁₃H₁₈N₃O₇⁺ | |
| 6 | 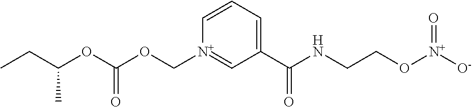 [SMILES: CC[C@@H](C)OC(=O)OC[n+]1cccc(C(=O)NCCO[N+](=O)[O-])c1] | Iodide |
| | C₁₄H₂₀N₃O₇⁺ | |
| 7 | 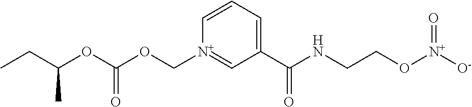 [SMILES: CC[C@H](C)OC(=O)OC[n+]1cccc(C(=O)NCCO[N+](=O)[O-])c1] | Iodide |
| | C₁₄H₂₀N₃O₇⁺ | |
| 8 | 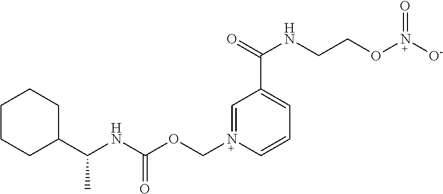 | Iodide |
| | C₁₈H₂₇N₄O₆⁺ | |
| 9 | 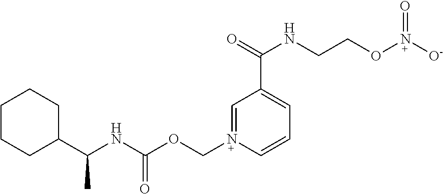 | Iodide |
| | C₁₈H₂₇N₄O₆⁺ | |
| 10 | 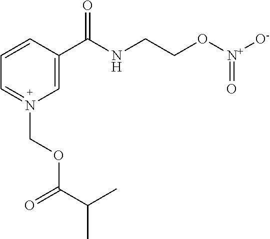 | Iodide |
| | C₁₃H₁₈N₃O₆⁺ | |
| 11 | 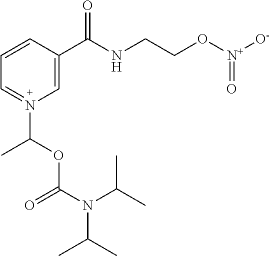 | Iodide |
| | C₁₇H₂₇N₄O₆⁺ | |
| 12 | 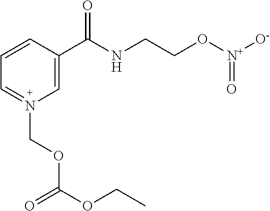 | Iodide |
| | C₁₂H₁₆N₃O₇⁺ | |
| 13 | 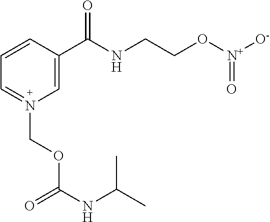 | Iodide |
| | C₁₃H₁₉N₄O₆⁺ | |

### Additional Exemplary Compounds

Other compounds provided herein have been or can be prepared according to the synthetic methods, or some variations thereof, described herein. The compounds can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The following additional compounds are or can be prepared from readily available starting materials using the following general methods and procedures:

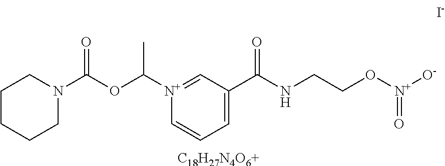

### Example 101 (PK)

### Pharmacokinetics (PK) Protocol: Dog

Three groups of beagle dogs were administered a single oral dose of 3, 10, or 30 mg/kg of Compound 8 ((S)-1-(((1-cyclohexylethylcarbamoyl)oxy)methyl)-3-((2-(nitroxy)ethyl)carbamoyl)pyridine-1-ium iodide). Clinical observations were recorded at approximately 1, 1.5, 2, 3, and 24 hour post-dose. Whole blood samples were collected pre-dose and 0.083, 0.25, 0.50, 1, 1.5, 2, 4, 8, and 24 hour post-dose to analyze systemic exposure to Compound 8 and nicorandil. Dose and concentration parameters (Cₘₐₓ and AUC) were used to generate linearity plots and calculate the coefficients of determinations (r²) and slopes for Compound 8 and nicorandil.

The mean Cₘₐₓ were 43, 178, and 1500 ng/mL for Compound 8. The Cₘₐₓ for nicorandil were 1300, 3070, and 7530 ng/mL. In the same three dosage groups, mean AUC were 34, 162, and 1510 h*ng/mL for Compound 8. The mean AUC were 3400, 114, and 38000 h*ng/mL for nicorandil. For linearity of dose vs. Cₘₐₓ, r² were 0.97 for Compound 8 and 1.00 for nicorandil.

Nicorandil was rapidly formed from the prodrug of Compound 8. Mean Cₘₐₓ for nicorandil was greater than 5-fold than that of Compound 8, demonstrating the efficient conversion of the prodrug to the active drug. The conversion was consistent across dose groups. These results indicate that Compound 8 is rationally designed drug and offers an opportunity to serve as an extended-release version of nicorandil. As a prodrug, Compound 8 may demonstrate improved GI tolerability, better overall safety profile, and longer half-life, which would make it a welcome addition to the treatment landscape. Future studies should evaluate Compound 8 in the target population of patients with or at risk of decompensated cirrhosis and alcoholic hepatitis.

### Example 102 (PK)

### Pharmacokinetics (PK) Protocol: Rat

Pharmacokinetics study was carried out to evaluate the plasma exposure of Compound 8 ((S)-1-(((1-cyclohexylethylcarbamoyl)oxy)methyl)-3-((2-(nitroxy)ethyl)carbamoyl)pyridine-1-ium iodide) in rat. The dosing vehicle used in this study was PEG400. 1,3.10 mg/kg oral dosing was done in overnight fasted SD rats and the parent drug generated was monitored. Also presence of modified drug was checked. After oral (PO) dosing blood was collected by serial bleeding at 8 different time points in heparinised tubes. Blood samples were centrifuged at 10,000 rpm for 5 min. at 4° C. to obtain the plasma, which were aspirated into separate labeled tubes and stored at −80° C. Extraction solvent was added to plasma, was vortexed and shaken on shaker for 10 minutes, centrifuged at 10,000 rpm for 10 minutes at 4° C. Supernatant was kept for analysis. Acetonitrile and plasma calibration curves were generated and percentage of drug recovery from plasma determined. Quantitative analysis was done by liquid chromatography tandem mass spectrometer (API3000 LC-MS/MS). Cₘₐₓ, Tₘₐₓ, AUC and t1/2 were calculated using Graph Pad PRISM version 5.04 and the results are depicted in Table 2.

TABLE 2
Pharmacokinetics parameters for Compound 8
(1, 3, 10 mg/kg oral dose in SD rats)

| Parameters | Compound 8 | Compound 8 | Compound 8 | Nicorandil |
|---|---|---|---|---|
| Rat PK | (1 mpk oral) | (3 mpk oral) | (10 mpk oral) | (3 mpk oral) |
| Cmax (nM) | 2529.42 | 9083.49 | 34705.8 | 7767.31 |
| Tmax (hr) | 2.00 | 1.17 | 2.0 | 1.17 |
| AUC (nM-hr) | 9788 | 46437.67 | 127639.0 | 20347.50 |
| Elimination | 2.28 | 3.67 | 2.1 | 3.43 |
| t_{1/2} (hr) | | | | |

### Example 201: Effect of Compound 8 on β2-Microglobulin (β2-MG) Excretion in Rats with Ischemia-Reperfusion (IR) Injury

Animal models of IRI (ischemia reperfusion injury), DGF (delayed graft function), and AKI (acute kidney injury) mimic many features seen in human kidney allografts. IRI induced rat/mouse models of AKI possess biphasic injuries (e.g., tubular injury followed by inflammatory reaction). In 2011, the FDA held a workshop to review the current state of knowledge related to the effects of IRI on outcomes in kidney transplantation in animal models. The FDA stated animal models have implicated maladaptive host responses to AKI in the genesis of IRI. The maladaptive responses are changes in hemodynamics, decrease in oxygen tension, mitochondrial dysfunction, tubular injury, inflammation, etc. Collectively, animal models have indicated that these maladaptive responses exacerbate injury and contribute to DGF. In conclusion, the FDA stressed the importance of investigations in animal models with established acute tubular injury, which mostly mimics IRI and DGF in humans. Hence, IRI induced rat model of AKI with acute tubular injury is used in the studies hereunder instead of animal models of DGF as there are experimental challenges with animal model of DGF.

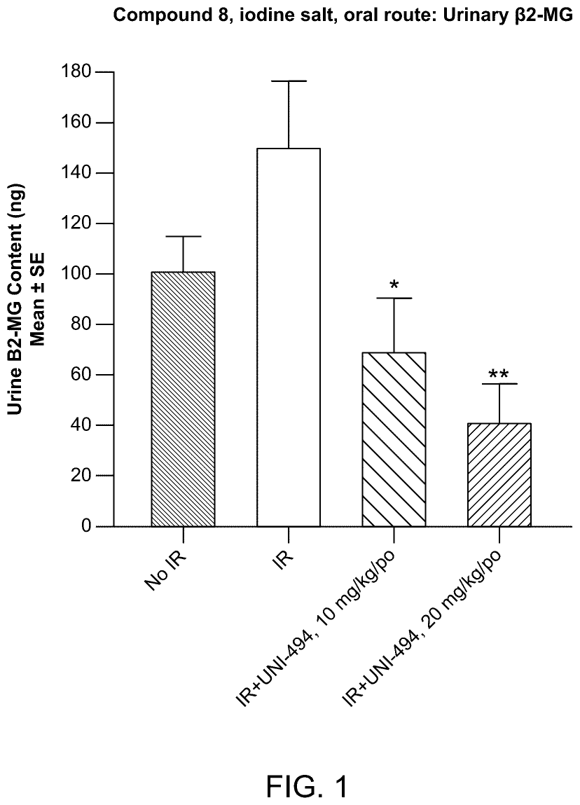

This study was conducted to evaluate the in vivo therapeutic efficacy of Compound 8 (iodide salt) administered orally to male Sprague Dawley rats (n=49), 1 hour prior to ischemic modelling, at doses of 10 mg/kg and 20 mg/kg using the rat bilateral ischemia/reperfusion (IR) model. In the rat bilateral IR model, the bilateral renal pedicle was clamped for 45 min for ischemia and then the clamp is removed to allow reperfusion. When compared with Group 1 (sham group—No IR), Group 2 (IR) and Groups 3 and 4 (IR with 10 and 20 mg/kg, respectively) exhibited significantly increased serum BUN and Cr levels. Urine β2-MG levels and content of Group 2 were higher than those in Group 1. Compared with Group 2, Groups 3 and 4 had lower urine β2-MG levels and content (n=10/group) (FIG. 1; p<0.5 at 10 mg/kg dose and p<0.01 at 20 mg/kg dose—as per one-way ANOVA multiple comparison test). Creatinine values and content of Groups 2 to 4 were significantly decreased in urine, whilst urine total albumin values were increased in these groups. Urine albumin-creatinine ratio (uACR) was also increased in Groups 2 to 4, but without statistical significance. The histological scores for tubular injury of all model groups (Groups 2 to 4) were significantly increased, indicating the successful establishment of the rat bilateral I/R model.

There was no significant difference between Compound 8 and vehicle groups in urine creatinine concentration or total amount, urine albumin concentration or total amount, or uACR. Similarly, no physical or behavioral abnormalities, significant changes in body weight, or significant differences in histological scores for tubular injury were observed between Compound 8 groups and vehicle.

Overall, Compound 8 did not appear to have a significant impact on creatinine or histological markers of kidney function at doses up to 20 mg/kg but did show reno-protective activity about $2-MG in this rat model of AKI. This study suggests that the pharmacologically active dose may be higher than 20 mg/kg.

### Example 202: Effect of Compound 8 on Kidney Functional Markers and Proximal Tubular Damage in Rats with Ischemia-Reperfusion Injury

The unilateral IR model of AKI in rats were used to study the efficacy of Compound 8 on kidney injury with a special focus on kidney functional markers (serum creatinine, sCre; blood urea nitrogen, BUN; and urinary albumin:creatinine ratio, ACR) and tubular injury markers (urinary niutrophil gelatinase-associated 1ipocalin, NGAL; and β2-microglobulin, β2-MG) and proximal tubular damage (proximal tubular injury scores via histology, Jablonski et al 1983). A total of 80 male SD (Sprague Dawley) rats were divided into 10 groups each with 10 rats/group as detailed in the Table 3. The ischemia was induced in the left kidney using a small clamp (Sugita standard aneurysm clip holding force 145 g) that was placed on the renal vascular pedicle for 30 min for all groups except SHAM group. For the SHAM group, sham operations were performed in a comparable manner, except that the renal vessels were not clamped for sham group (No IR group).

Compound 8, nitrate salt, was administered 30 minutes prior to the induction of ischemia via oral (po) administration or intraperitoneal (ip) administration or intravenous (iv) administration to group numbers 3 to 8 as detailed in Table 3. Compound 8, nitrate salt, was dosed intravenously at 1 or 4 hours after ischemia, i.e., at therapeutic mode as detailed in the groups 9 and 10 of the Table 3.

TABLE 3
Details about the dose regimen

| Group and | Group and | | | Duration | Duration | | | | | | | # of |
|---|---|---|---|---|---|---|---|---|---|---|---|---|
| Route of | Route of | Group | | of | of | First | | Dosage | | | Time of | rats/ |
| administration | administration | Number | Group | Ischemia | Reperfusion | dose at | | mg/kg | | Dose | Termination | group |
| Preventive | NO IR | 1 | SHAM | 30 minutes | 24 hours | −30 | minutes | 0 | | Vehicle | 24 hours | 10 |
| Regimen | IR | 2 | Vehicle | 30 minutes | 24 hours | −30 | minutes | 0 | | Vehicle | 24 hours | 10 |
| | IR + Oral | 3 | Compound 8 | 30 minutes | 24 hours | −30 | minutes | 50 | mg | Low dose | 24 hours | 10 |
| | | 4 | Compound 8 | 30 minutes | 24 hours | −30 | minutes | 100 | mg | High dose | 24 hours | 10 |
| | IR + | 5 | Compound 8 | 30 minutes | 24 hours | −30 | minutes | 25 | mg | Low dose | 24 hours | 10 |
| | Intraperitoneal | 6 | Compound 8 | 30 minutes | 24 hours | −30 | minutes | 50 | mg | High dose | 24 hours | 10 |
| | IR + | 7 | Compound 8 | 30 minutes | 24 hours | −30 | minutes | 5 | mg | Low dose | 24 hours | 10 |
| | Intravenous | 8 | Compound 8 | 30 minutes | 24 hours | −30 | minutes | 10 | mg | High dose | 24 hours | 10 |
| Therapeutic | IR + | 9 | Compound 8 | 30 minutes | 24 hours | 1 | hour | 10 | mg | High dose | 24 hours | 10 |
| Regimen | Intravenous | 10 | Compound 8 | 30 minutes | 24 hours | 4 | hours | 10 | mg | High dose | 24 hours | 10 |

All rats were housed in metabolic cages for the collection of 24-hour urine samples for ACR, NGAL and β2-MG levels. After 24 hours, rats were anaesthetized, and blood samples were collected for sCre, and BUN levels. The left kidney was collected and processed for histology for tubular injury scores.

In this study, IR induced significant increases of sCr, BUN, ACR, NGAL, β2-MG, and proximal tubular injury damage scores in the vehicle treated IR group when compared to No IR sham group (p<0.0001—as per one-way ANOVA multiple comparison test).

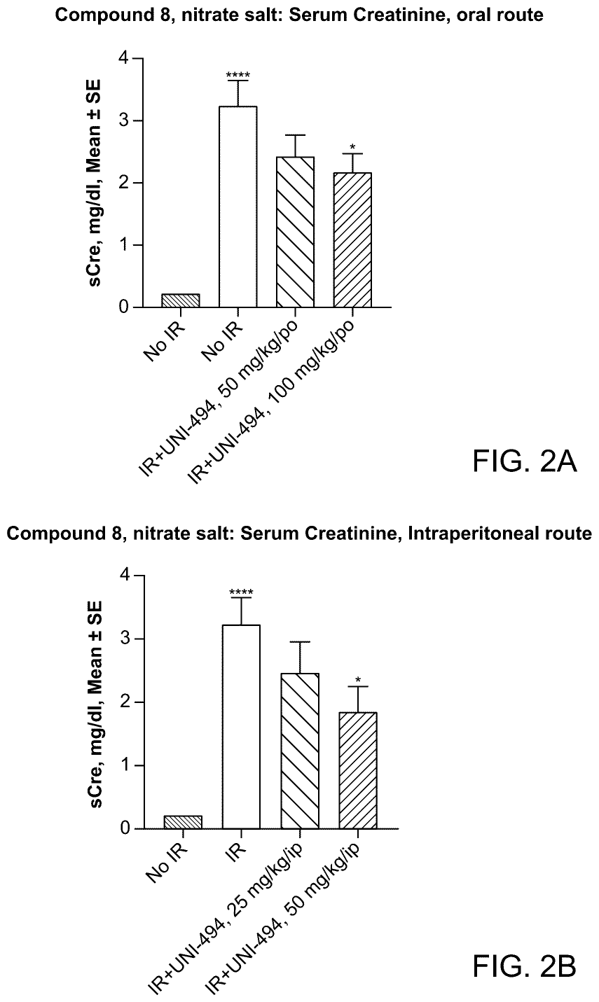

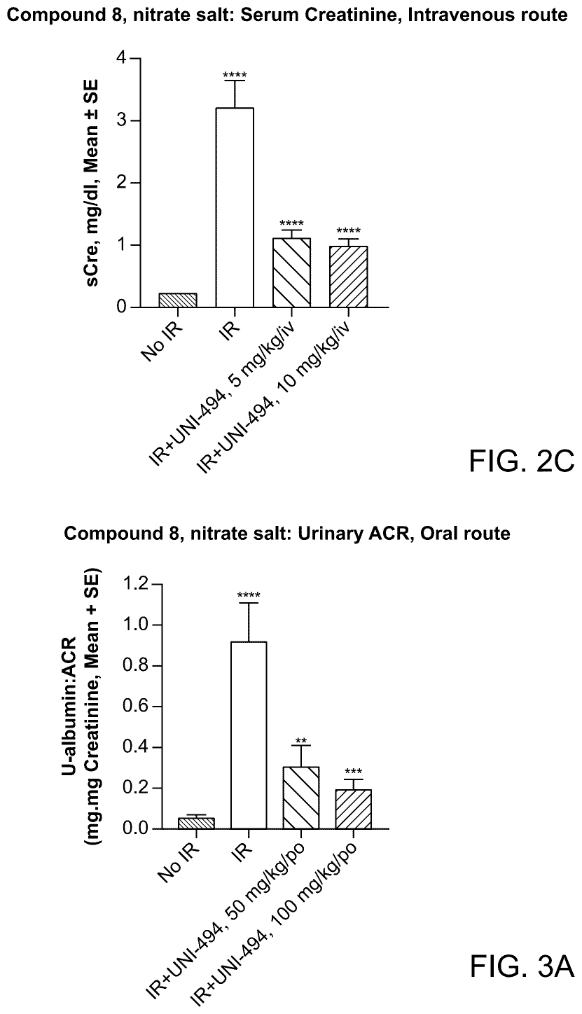

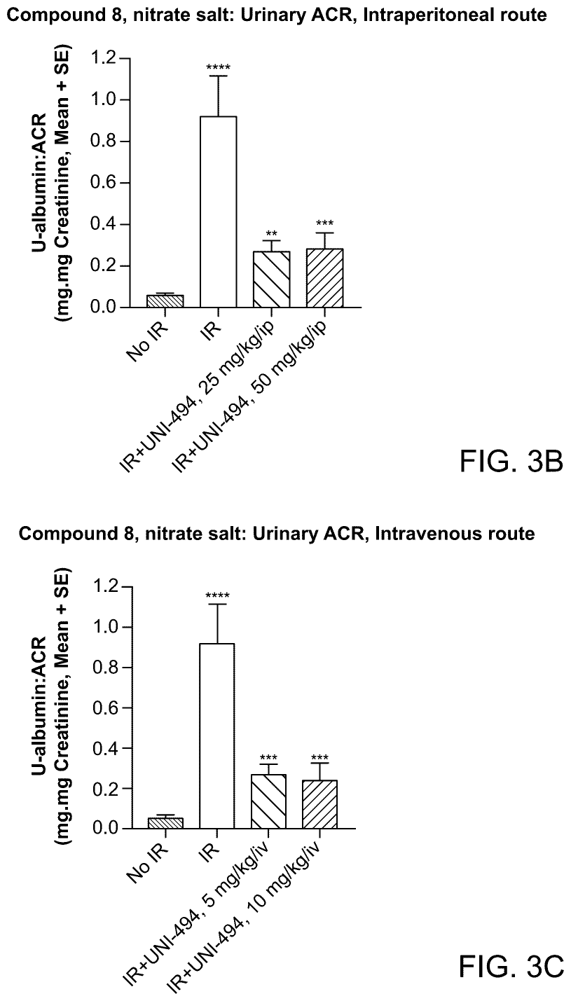

Compound 8, nitrate salt, exhibits reduction of kidney functional markers. Compound 8, nitrate salt, in oral route of administration: A single dose of Compound 8 at 50 mg/kg/po (low dose) or 100 mg/kg/po (high dose), administered po route, significantly reduced the kidney functional markers serum creatinine (p<0.05 at high dose—FIG. 2A); and ACR (low dose p<0.01; high dose p<0.001—FIG. 3A).

Compound 8, nitrate salt, in intraperitoneal route of administration: A single dose of Compound 8 at 25 mg/kg/ip (low dose) or 50 mg/kg/ip (high dose), administered ip, significantly reduced the kidney functional markers serum creatinine (p<0.05 at high dose—FIG. 2B); and ACR (low dose p<0.01; high dose p<0.001—FIG. 3B).

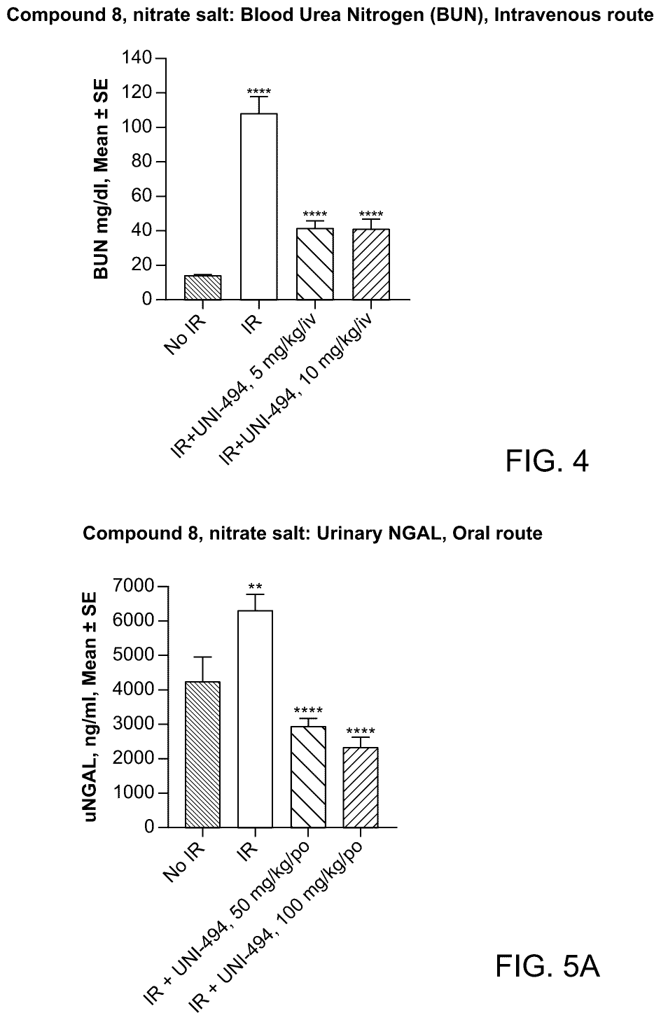

Compound 8, nitrate salt, in intravenous route of administration: A single dose of Compound 8 at 5 mg/kg/iv (low dose) or 10 mg/kg/iv (high dose), administered intravenously (iv), phenomenally reduced the kidney functional markers serum creatinine (p<0.0001 at low dose and p<0.0001 at high dose—FIG. 2C); BUN (p<0.0001 at low dose and p<0.0001 at high dose—FIG. 4) and ACR (p<0.001 at low dose; p<0.001 at high dose—FIG. 3C).

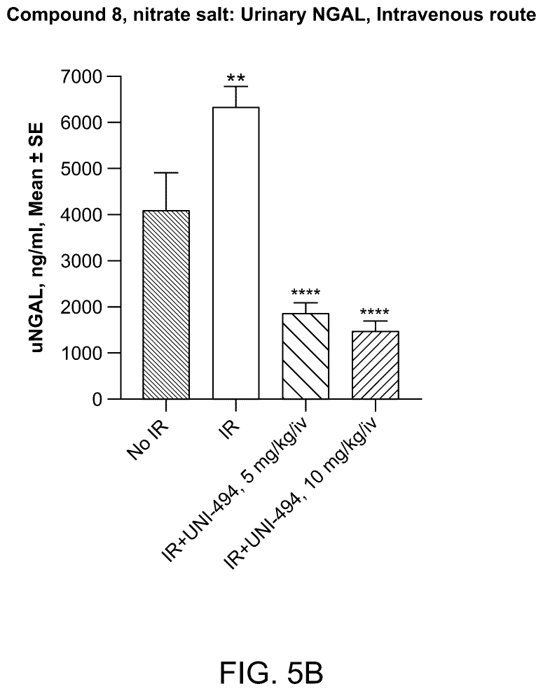

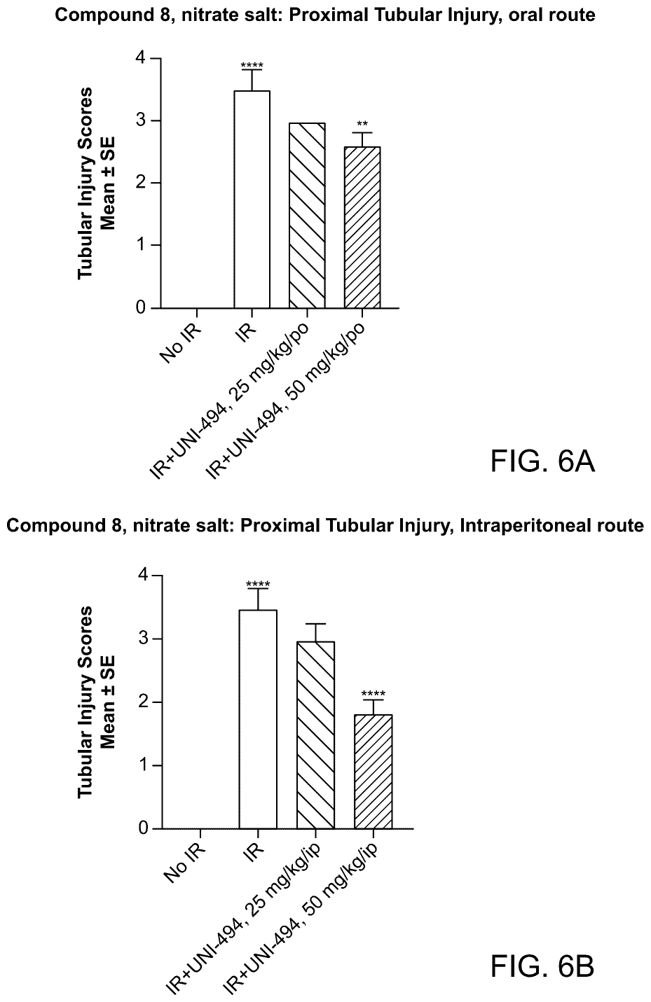

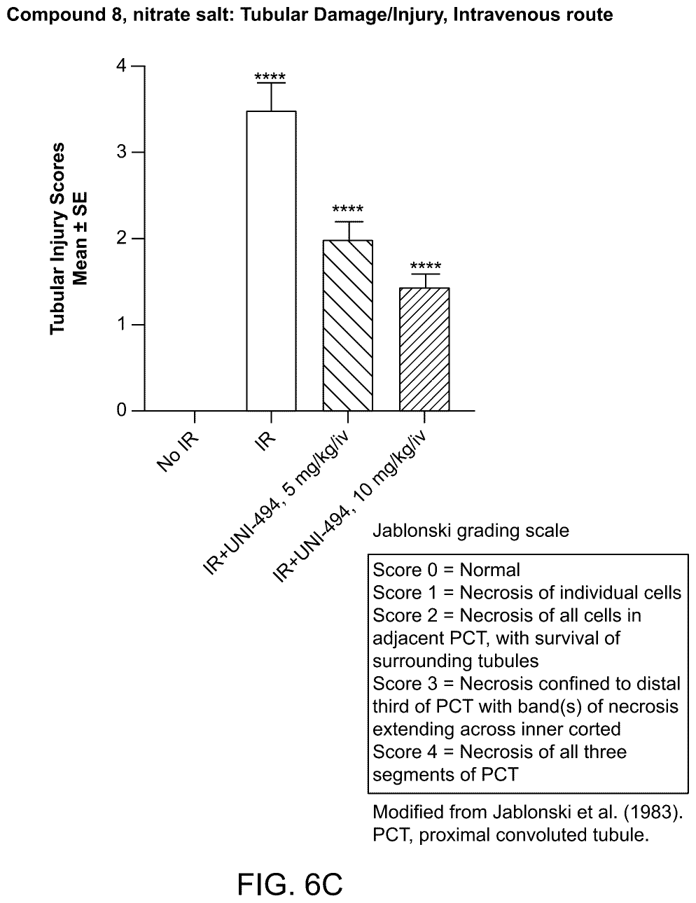

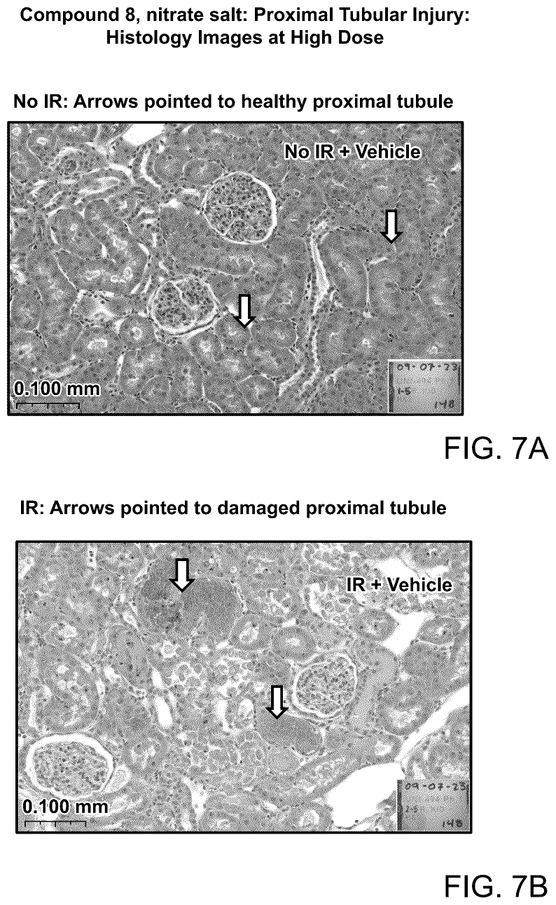

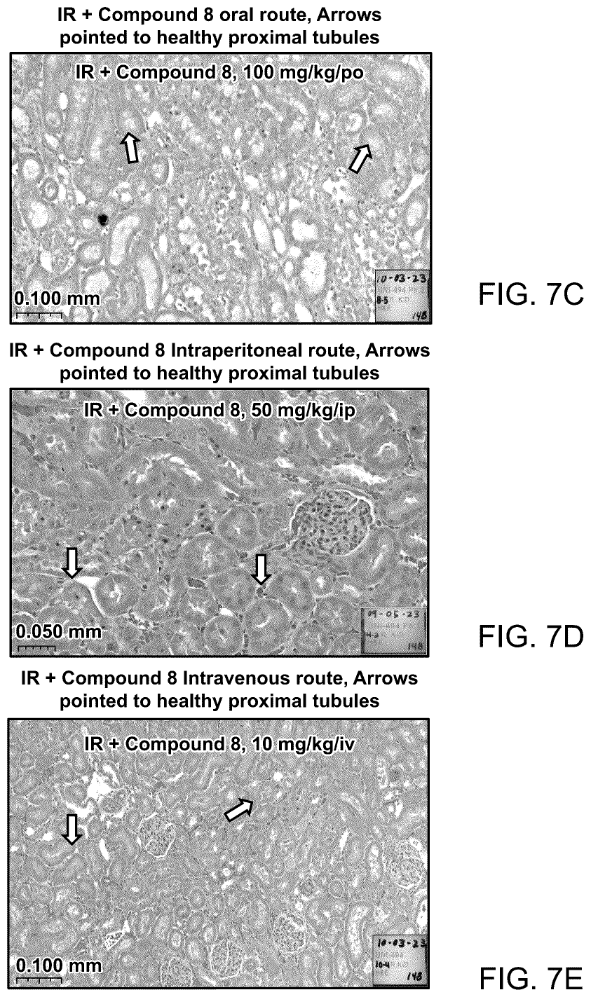

Compound 8, nitrate salt, exhibits reduction of tubular injury makers/damage. Compound 8, nitrate salt, in oral route of administration: Compound 8 was effective in lowering tubular injury marker NGAL at the low dose (p<0.0001 as well as at high dose p<0.0001—FIG. 5A) and proximal convoluted tubular injury scores/damage at high dose (p<0.0001—FIG. 6A). Histological images were presented in FIGS. 7A-7E. The proximal tubules are significantly damaged which is evidenced by necrotic and denuded epithelial cells in IR+vehicle treated group (FIG. 7B) when compared to the No IR/SHAM group (FIG. 7A). Compound 8, nitrate salt, moderately ameliorated tubular injury/damage (FIG. 7C—pointed healthy tubules with arrows).

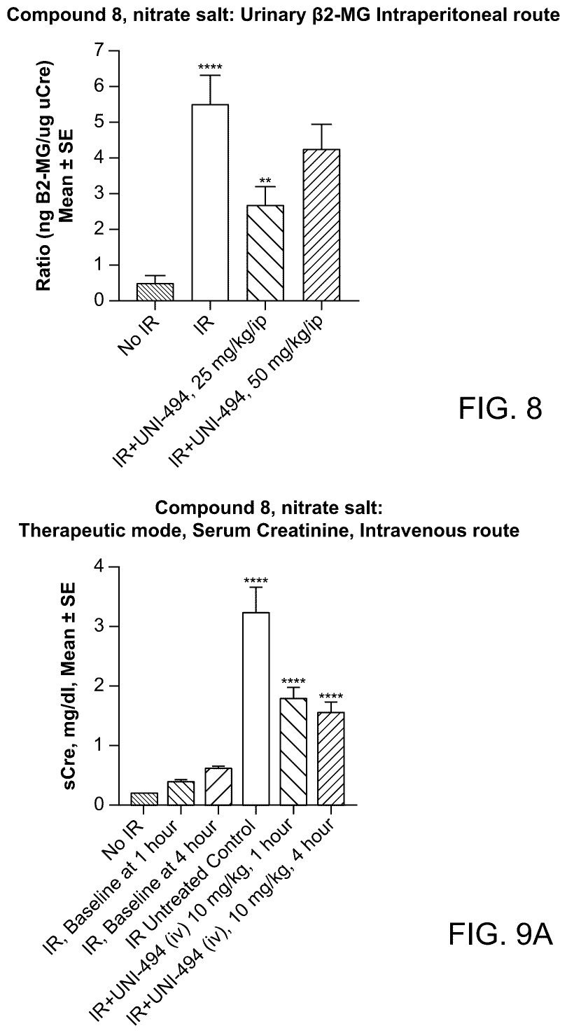

Compound 8, nitrate salt, in intraperitoneal route of administration: Compound 8, nitrate salt, was effective in lowering tubular injury marker β2-MG at the low dose (p<0.01—FIG. 8) and proximal convoluted tubular injury scores at high dose (p<0.0001—FIG. 6B). Histological images were presented in FIGS. 7A-7E. The proximal tubules are significantly damaged which is evidenced by necrotic and denuded epithelial cells in IR+vehicle treated group (FIG. 7B) when compared to the No IR/SHAM group (FIG. 7A). Compound 8, nitrate salt, moderately ameliorated tubular injury/damage (FIG. 7D—pointed healthy tubules with arrows).

Compound 8, nitrate salt, in intravenous route of administration: Compound 8, nitrate salt, was robustly effective in lowering tubular injury marker NGAL at the low dose (p<0.0001 and high dose p<0.0001—FIG. 5B) and proximal convoluted tubular injury scores at both low dose (p<0.0001) and at high dose (p<0.0001—FIG. 6C). Histological images were presented in FIGS. 7A-7E. The proximal tubules are significantly damaged which is evidenced by necrotic and denuded epithelial cells in IR+vehicle treated group (FIG. 7B) when compared to the No IR/SHAM group (FIG. 7A). Compound 8, nitrate salt, markedly ameliorated tubular injury/damage and the data is statistically highly significant at both low and high doses (FIG. 6C). The histological image at high dose was presented as FIG. 7E where most all proximal tubules are healthy—pointed with arrows). The data at 5 mg/kg/iv demonstrated maximum benefit and follow up studies with a dose under 5 mg/kg/iv are in progress.

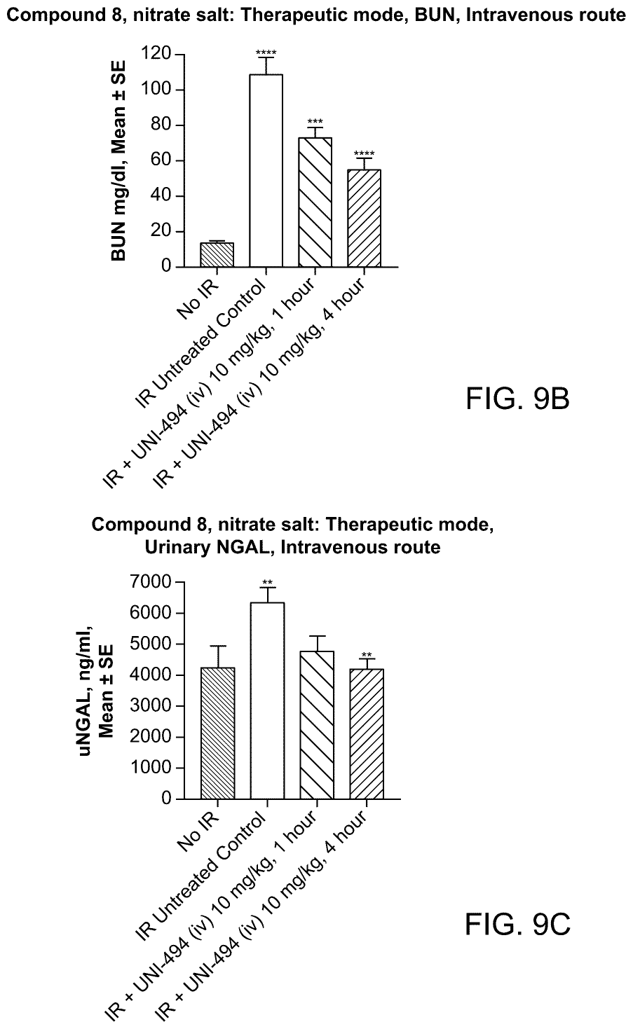

Compound 8, nitrate salt, in intravenous route of administration in a therapeutic regimen: As detailed in FIG. 9A, serum creatinine was elevated after IRI i.e., at baseline at 1 and 4 hours timepoints when compared to the 0 hours time point i.e., before IRJ. It suggests IRI induced renal injury is established by 1 hour and 4 hours timepoints. At 1 hour and 4 hours timepoints with established IR injury (i.e., at therapeutic mode), a single dose of Compound 8 at 10 mg/kg/iv (high dose) administered intravenously (iv), phenomenally reduced the kidney functional markers serum creatinine at both 1 hour and 4 hours (p<0.0001; FIG. 9A), BUN at 1 hour (p<0.001; FIG. 9B) and 4 hours (p<0.0001: FIG. 9B); and urinary NGAL at 4 hours (p<0.01; FIG. 9C).

It is concluded that Compound 8, nitrate salt, prevents ischemia induced renal injury when it is administrated oral, or intraperitoneal or intravenous routes. In addition, compound 8 reverses IRI when it is dosed intravenously in an established renal injury state i.e., at therapeutic mode; and it is a potential candidate for the treatment of patients with DGF and related clinical conditions dealt with acute kidney injury. Compound 8, nitrate salt, demonstrated a much superior effect on ameliorating renal injuries when it is administered intravenous route when compared to other route of administration.

The data from these two studies clearly indicate Compound 8 (both in the nitrate and iodine salt formations) dose dependently ameliorates IR injury (tubular injury) in rat model of AKI via halting mitochondrial dysfunction. This data has the greatest clinical potential in the context of DGF where IR injury is an inevitable consequence of kidney transplant and has been correlated with the incidence of acute renal transplant rejection.

### Example 203: Effect of Nicorandil on Kidney Functional Markers and Proximal Tubular Damage in Rats with Ischemia-Reperfusion Injury

The unilateral IR model of AKI in rats were used to study the efficacy of Compound 8 nitrate salt's parent compound “Nicorandil” on kidney injury with a special focus on kidney functional markers (serum creatinine, sCre; and urinary albumin:creatinine ratio, ACR) and proximal tubular damage (proximal tubular injury scores via histology, Jablonski et al 1983). A total of additional 10 male SD (Sprague Dawley) rats were used along with the Group 1 (No IR) and Group 2 (IR); and other Groups listed in the Tabel 3; and conducted the study. The ischemia was induced in the left kidney of those additional 10 rats using a small clamp (Sugita standard aneurysm clip holding force 145 g) that was placed on the renal vascular pedicle for 30 min.

Nicorandil at 10 mg/kg, was administered 30 minutes prior to the induction of ischemia via intraperitoneal (ip) administration to the additional 10 rats along with the groups listed in Table 3. All additional 10 rats were housed in metabolic cages for the collection of 24-hour urine samples for ACR. After 24 hours, rats were anaesthetized, and blood samples were collected for sCre. The left kidney was collected and processed for histology for tubular injury scores along with the other groups as detailed in Table 3.

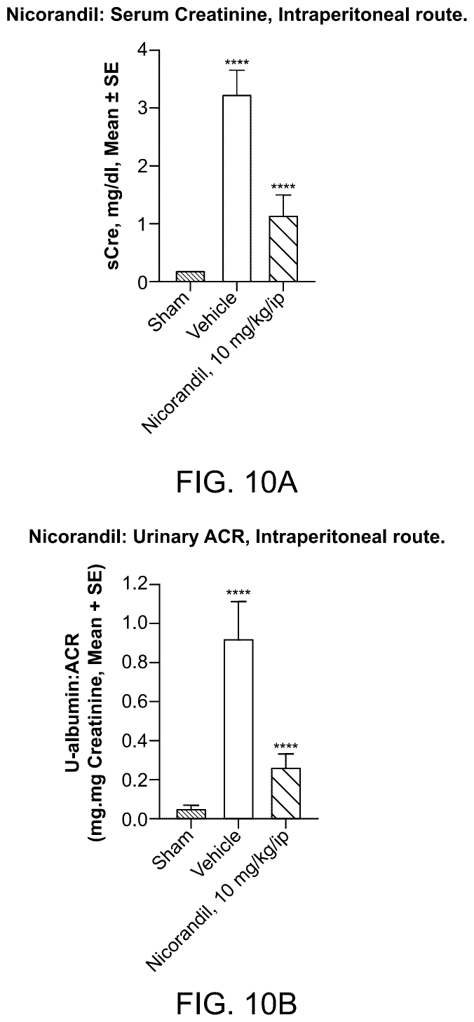

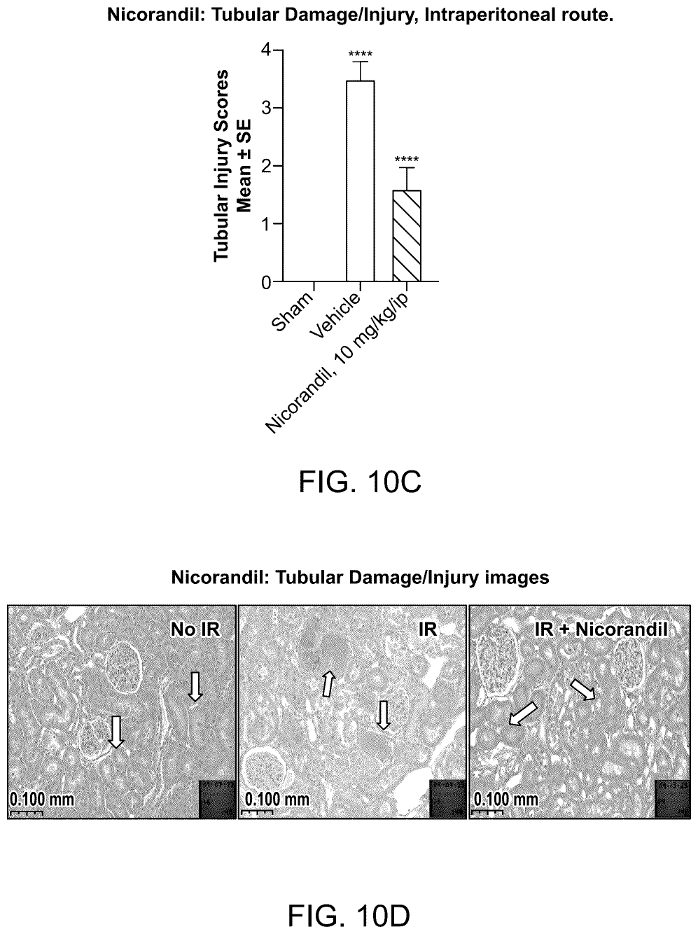

Nicorandil, in intraperitoneal route of administration: A single dose of nicorandil at 10 mg/kg, administered ip, significantly reduced the kidney functional marker serum creatinine (p<0.0001—FIG. 10A), ACR p<0.0001—FIG. 10B) and tubular injury/damage (p<0.0001—FIG. 10C) when compared to the Group 2 (No IR). The histological images were presented as FIG. 10D with 3 panels No IR, IR and Nicorandil. The most all proximal tubules are healthy with Nicorandil treated group—pointed with arrows).

Like Compound 8 nitrate salt, parent compound nicorandil also ameliorates IR injury (tubular injury) in rat model of AKJ. The present example demonstrates that nicorandil useful for the prevention, treatment, and/or amelioration of DGF.

### Example 301: Effect of Compound 8 on Kidney Functional Markers in Kidney Transplant Patients at High Risk of Delayed Graft Function (DGF)

This is a within-group randomized, double-blinded, placebo-controlled, sequential-group, multicenter, Phase 1b study to evaluate the safety and tolerability of Compound 8 in patients receiving a kidney transplant. The Screening Period (Day −28 to Day −1) lasts up to up to 28 days prior to the first dose on Day 1 on the day of transplant. Patients must meet all Inclusion/Exclusion Criteria to participate in the study. Eligible patients will provide written Informed Consent prior to any study procedures. Eligible patients in each group will be randomized to receive either a single dose of Compound 8 or placebo in Group 1 or receive Compound 8 twice daily (BID) for 5 days in Groups 2 and 3. Enrollment of the next higher dose will begin following a review of the safety data from the prior dose by an independent data monitoring committee (DMC).

The doses are based on the safe doses identified in the Phase 1 study in healthy subjects (Compound 8). The three groups are:
Group 1: 160 mg single dose of Compound 8 4-6 hours prior to transplantation—12 patients (9 active, 3 placebo)
Group 2: 40 mg BID for 5 days with the first dose beginning 4-6 hours prior to transplantation and then for 4 days after transplantation—12 patients (9 active, 3 placebo)
Group 3: 80 mg BID for 5 days with the first dose beginning 4-6 hours prior to transplantation and then for 4 days after transplantation—12 patients (9 active, 3 placebo)

The following kidney specific assessments will be made in patients for up to 5 days.
24-hour urine
Kidney specific biomarkers: Beta-2 Microglobulin (B2M) Blood Urea Nitrogen (BUN)
Kidney Injury Molecule 1 (KIM1), Neutrophil Gelatinase-Associated Lipocalin (NGAL), Serum Creatinine (SCr)
Need for dialysis within the first 7 days after transplant

### Inclusion & Exclusion Criteria:

### Inclusion Criteria:

1. Patient is at least 18 years of age.
2. Patient must be willing and capable of giving written Informed Consent, adhering to dosing and visit schedules, and meeting all study requirements.
3. Patient is eligible and receiving a kidney transplant per site specific guidelines.
4. Patients must be willing and able to swallow multiple capsules.
5. Females of childbearing potential must have a negative pregnancy test within 7 days prior to the first dose on Day 1.
6. Patient is willing to practice 2 forms of contraception, one of which must be a barrier method, from study entry until at least 30 days after the last dose of Compound 8.

### Exclusion Criteria:

1. Patient is planned to receive a multi-organ transplant.
2. Patient has had a serious adverse reaction or serious hypersensitivity reaction to nicorandil or any formulation excipients.
3. Patients with any history of GI ulceration or GI hemorrhage within 6 months prior to the first dose on Day 1.
4. Recurrent and recent history of simple faints, vasovagal presyncope/syncope or blackouts.
5. Patients with a history of conjunctival or corneal ulceration within the past 12 months.
6. Patient has clinically significant abnormal clinical chemistry or hematology as judged by the Investigator. Patients with Gilbert's Syndrome are not allowed.
7. Patient has a positive hepatitis B surface antigen (HBsAg), hepatitis C virus antibody (HCV Ab) or human immunodeficiency virus (HIV) 1 and 2 antibody results.
8. Patient has received any investigational product in a clinical research study within the 90 days prior to the first dose on Day 1, or less than 5 elimination half-lives prior to the first dose on Day 1, whichever is longer.
9. Patient is using PDE5 inhibitors.
10. Patient is unable to take drugs orally due to disorders or diseases that may affect gastrointestinal function, such as inflammatory bowel diseases (eg, Crohn's disease, ulcerative colitis) or malabsorption syndrome, or procedures that may affect gastrointestinal function, such as gastrectomy, enterectomy, or colectomy.
11. Patient has an active uncontrolled infection, underlying medical condition, or other serious illness that would not be appropriate for this study.
12. Patient is pregnant or breast-feeding.

Compound 8 is supplied as identical looking 5 mg and 15 mg capsules and will be taken by the patient orally on the following schedule:
Group 1: single dose 4-6 hours prior to transplantation
Groups 2 and 3: twice daily for 5 days with the first dose administered 4-6 hours prior to transplantation on Day 1 and then for 4 days

### Efficacy Assessments:

### Primary Endpoint:

Discontinuations due to treatment-related AEs
Major Adverse Kidney Events within 30 days (MAKE-30)
Frequency and severity of adverse events (AEs) and serious AEs

### Secondary Endpoints:

Kidney-function specific biomarkers (Serum Creatinine (SCr, urine creatinine, BUN)
Kidney-injury biomarkers (NGAL, KIM-1)
Need for dialysis within the first 7 days after transplant
Pharmacokinetics of Compound 8 and its metabolites (nicorandil and 1-cyclohexylethylamine).

### Safety Endpoints

Safety will be assessed by reported/elicited adverse events (AEs), laboratory assessments including hematology, biochemistry, and urinalysis, vital signs, physical examination, electrocardiograms, and neurological examination. The assessment of treatment-emergent AEs (TEAEs) includes SAEs, AEs leading to study drug discontinuation, and AEs related to the study drug.

### Pharmacokinetic and Biomarker Endpoints

Group 1: Blood samples for intensive PK analysis will be collected on Day 2, approximately 24 (±2) hours after the dose of study drug was administered at 0.5, 1, 2, 4, 8, and 24 hours.
Group 2 and Group 3: Blood samples for intensive PK analysis will be collected on Day 5 at predose (prior to the first daily dose) and 0.5, 1, 2, 4, 8, and 24 hours postdose.
Pharmacokinetic parameters of Compound 8, nicorandil, and 1-cyclohexylethylamine will include: plasma half-life (t_{1/2}), maximum plasma concentration (Cₘₐₓ), time to Cₘₐₓ (Tₘₐₓ), area under the curve from time zero to 24 hours (AUC₀₋₂₄), and area under the curve from time zero to infinity (AUC_{0-inf}).

### Analysis Populations

The following analysis populations have been defined:
The Evaluable Population will be the primary analysis population and include all patients who receive at least one dose of Compound 8 or placebo. Biomarker assessments will be made using the Evaluable Population.
The Safety Population includes all patients who signed Informed Consent, enrolled, and received at least one dose of Compound 8 or placebo. All demographics, Baseline characteristics, and safety data will be analyzed using the Safety Population.
The Pharmacokinetic (PK) Population will include all subjects with at least one available valid (i.e. not flagged for exclusion) PK concentration measurement, who received any study drug and experienced no protocol deviations with relevant impact on PK data.

### Example 401: Effect of Compound 8 on Kidney Functional Markers in Kidney Transplant Patients at High Risk of Delayed Graft Function (DGF)

In this Phase I study, the safety and tolerability of single ascending doses (SAD) of Compound 8 was administered to male and female subjects. The study was a single centre, double-blinded, placebo-controlled, randomized single ascending doses (SAD) and multiple ascending doses (MAD) study in healthy male and female subjects of non-childbearing potential.

Part 1 was a double-blinded, randomized, placebo-controlled, SAD study, including approximately 32 subjects in 4 cohorts of 8 subjects each (randomized to a ratio of 6 active and 2 placebo per cohort). The subjects received the regimens presented in Table A. The route of administration was oral and fasted. IMP refers to investigated medicinal product.

TABLE A

| Cohort | Period | Regimen | IMP | Dose |
|---|---|---|---|---|
| SAD 1 | N/A | A | Compound 8 | 10 mg |
| | | | Capsule or | |
| | | | Placebo | |
| SAD 2 | N/A | B | Compound 8 | 20 mg |
| | | | Capsule or | |
| | | | Placebo | |
| SAD 3 | 1 | C | Compound 8 | 40 mg |
| | | | Capsule or | |
| | | | Placebo | |
| SAD 4 | N/A | D | Compound 8 | 80 mg |
| | | | Capsule or | |
| | | | Placebo | |
| SAD 5 | 2 | E | Compound 8 | 160 mg |
| | | | Capsule or | |
| | | | Placebo | |

The maximum allowed dose escalation was 2-fold more than the previous dose. The dose selected considered the individual data such that the highest exposure in any individual would not be predicted to exceed the safety exposure limits of Compound 8 or nicorandil:
Compound 8: Cₘₐₓ 54.2 ng/mL and AUC(0-last)=39.8 ng·h/mL;
Nicorandil: Cₘₐₓ=621 ng/mL and AUC(0-last)=1300 ng·h/mL.

Subjects received a single dose of Compound 8 Capsule or placebo in the morning following an overnight fast. For Regimen E, Period 2, cohort SAD 3 were readmitted to the clinical unit. There was a minimum 7-day washout between dosing occasions for Regimen C and Regimen E. Blood samples were collected at regular intervals for safety and PK analysis from Day −1 to discharge from the clinical unit.

Part 2 of the study was a double-blind, randomized, placebo-controlled, MAD study. The subjects received the regimens presented in Table B. The route of administration was oral and fasted.

TABLE B

| Cohort | Regimen | IMP | Dose | Dosing Schedule |
|---|---|---|---|---|
| MAD 1 | F | Compound 8 | 80 mg | BID (4 days, and |
| | | Capsule or | | once in morning |
| | | placebo | | of Day 5) |
| MAD 2 | G | Compound 8 | 40 mg | BID (4 days, and |
| | | Capsule or | | once in morning |
| | | placebo | | of Day 5) |

Dose level and initiation of MAD Cohort 2 were selected based on emerging safety, tolerability and available PK data from SAD cohorts in Part 1 and also review of all available safety, tolerability and PK data from all subjects through at least Day 8 of prior MAD cohorts. The total daily dose of 80 mg was deemed to be acceptable from a safety perspective as this is a lower dose than what was previously administered in MAD Cohort 1, with adequate single dose coverage from Part 1.

The dose was chosen to ensure that the predicted exposures at steady state, was not to exceed those found to be safe and tolerated in Part 1 of the study, and that the highest exposure in any single individual did not exceed the safety exposure limits of Compound 8 or nicorandil:
Compound 8: Cₘₐₓ=54.2 ng/mL and AUC(0-tau)=39.8 ng·h/mL;
Nicorandil: Cₘₐₓ=621 ng/mL and AUC(0-tau)=1300 ng·h/mL.

Subjects received BID dosing of Compound 8 Capsule or placebo for 4 days, and one dose on Day 5. Subjects were dosed in the fasted state and were dosed in the morning and evening (approximately 12 hours apart) of Days 1 to 4 and on the morning of Day 5.

During dosing of Part 1, Cohort 1, subjects were administered single doses of 10 mg Compound 8 Capsules (2×5 mg) or matching placebo in the fasted state. The maximum individual Cₘₐₓ and AUC(0-last) values for Compound 8 exceeded the Compound 8 exposure limits defined in Version 2.0 of the protocol (Cₘₐₓ of 1.01 ng/mL and AUC(0-last) of 0.127 ng·h/mL) and the study was temporarily paused. Based on the maximum allowed escalations, the highest anticipated dose was to be 100 mg. The nicorandil equivalent dose of 100 mg Compound 8 is 40.4 mg nicorandil. This is approximately 60% of the NOAEL calculated human Compound 8 dose in the most relevant species (dog), and equal to the usual prescribed daily dose equivalent (20 mg BID), and half the maximum prescribed daily dose equivalent (40 mg BID) of the marketed drug nicorandil, which is the active metabolite of Compound 8. Although the highest anticipated dose was 100 mg, if exposures were below expectations and the defined exposure limits were not predicted to be exceeded, then dose escalation could proceed above 100 mg. This eventuality occurred, and thus following no safety concerns being raised at the 80 mg dose level and the determination that the increase in dose would not be expected to exceed the PK exposure limits, it was decided that the dose for Regimens E and F were to be 160 mg.

During dose escalation, no dose selected was expected to result in an exposure in any individual subject greater than the following for Compound 8 or nicorandil:
Compound 8: Cₘₐₓ=54.2 ng/mL and AUC(0-last)=39.8 ng·h/mL
Nicorandil: Cₘₐₓ=621 ng/mL and AUC(0-last)=1300 ng·h/mL.

In Part 1 of the study, subjects received a single oral doses of Compound 8 Capsule or placebo on one occasion in the fasted state, except for subjects in Cohort SAD 3 who received two single doses on two separate occasions. Doses ranged from 10 mg to 160 mg. In Part 2 of the study, subjects received single doses of Compound 8 Capsule or placebo BID for 4 consecutive days and once on Day 5 in the fasted state. Doses ranged from 40 mg to 80 mg.

### Pharmacokinetic and Statistical Conclusions for Compound 8

The absorption in part 1 of the study of Compound 8 was rapid over the 10 mg to 160 mg dose range, with median Tₘₐₓ occurring between 0.5 h and 1.5 hours post-dose. Where calculable, the individual plasma half-life of Compound 8 was short, ranging from 0.74 h to 2.58 hours over the entire dose range; however, it should be noted that reliable T_{1/2} results were only obtained from 11 out of the 30 profiles, with fewer than 3 estimates at all dose levels except 80 mg.

Results of the formal statistical analysis of dose proportionality indicated a sub-proportional increase in both peak and overall exposure with increasing dose over the 10 mg to 160 mg dose range, based on Cₘₐₓ and AUC(0-last). Peak and overall exposure, based on geometric mean Cₘₐₓ and AUC(0-last) appeared similar between the 80 mg and 160 mg dose levels; however, it should be noted that variability based on geometric CV % was high for these parameters, ranging between 46.0% and 102.8%.

### Pharmacokinetic and Statistical Conclusions for Nicorandil

In part one of the study, between the 20 mg and 160 mg dose levels, nicorandil appeared within the plasma with a median Tₘₐₓ of 4 hours post-dose. Plasma T_{1/2} was reported for one subject at the 160 mg dose level, with a result of 1.77 hours.

Exposures of active metabolite nicorandil were substantially greater than that of the pro-drug Compound 8. Based on geometric mean metabolite to parent ratios (nicorandil/Compound 8), molecular weight (MW)-corrected Cₘₐₓ values were between 31.4 and 150.9-fold higher than those of Compound 8 across the 20 to 160 mg dose range. MW-corrected AUC(0-last) values were between 65.8 to 209.0-fold higher across the 40 mg to 160 mg dose range. Results of the formal statistical analysis indicated that both peak exposure, over the dose range 20 mg to 160 mg, and total exposure, over the dose range 40 mg to 160 mg, increased in a slightly greater than proportional manner with increasing dose, based on Cₘₐₓ and AUC(0-last).

Pharmacokinetic and Statistical Conclusions for 1-cyclohexylethylamine

In part 1 of the study, PK profiles for 1-cyclohexylethylamine were entirely BLQ for the 10 mg, 20 mg and 40 mg dose levels with quantifiable concentrations observed in 4 of the 6 subjects at the 80 mg dose level. At the 160 mg dose level, quantifiable concentrations were observed in all subjects.

1-cyclohexylethylamine appeared to form slowly with Tₘₐₓ occurring between 10 hours and 16 hours post-dose and median Tₘₐₓ occurring at 12 hours post-dose for the both the 80 mg and 160 mg dose levels.

### Pharmacokinetic and Statistical Conclusions for Compound 8

The absorption in part 2 of the study of Compound 8 was rapid following a single and multiple doses of Compound 8 at 40 mg and 80 mg, with median Tₘₐₓ occurring at 1 to 1.5 hours post-dose. Results of the formal statistical analysis of dose-proportionality indicated an approximate proportional increase in exposure from the 40 mg to the 80 mg dose based on Cₘₐₓ and AUC(0-tau) on Day 1. Following multiple oral doses of Compound 8 at 40 mg and 80 mg for 5 days, the absorption of Compound 8 was rapid over both doses with median Tₘₐₓ occurring at 1.5 hours and 1 hour post-dose, respectively. Where calculable, the individual plasma half-life of Compound 8 on Day 5 was short, ranging from 1.11 hours to 1.42 hours over both doses, however, it should be noted that reliable T_{1/2} results were only obtained from 3 out of the 10 profiles. Following multiple doses, exposure to Compound 8 on Day 5 (based on geometric mean Cₘₐₓ and AUC(0-tau)) increased between the 40 mg and 80 mg doses, however it should be noted that variability based on geometric CV % was high for these parameters following the 40 mg dose, ranging between 69.4% and 73.5%.

Results of the formal statistical analysis of dose-accumulation indicated accumulation was seen by Day 5 following the 40 mg dose with GMRs of 1.30 for Cₘₐₓ and 1.85 for AUC(0-tau). No formal assessment of dose accumulation was performed following 80 mg dosing due to the low number of reliable results owing to multiple subject withdrawals prior to Day 5.

### Pharmacokinetic and Statistical Conclusions for Nicorandil

In part 2 of the study, following single and multiple doses of Compound 8 at 40 mg and 80 mg, nicorandil appeared within the plasma with a median Tₘₐₓ ranging between 3.5 hours and 4 hours post-dose, approximately 2.5 to 3.0 hours later than Compound 8. Results of the formal statistical analysis of dose-proportionality indicated a proportional increase in exposure from the 40 mg to the 80 mg dose based on Cₘₐₓ and AUC(0-tau) on Day 1. Exposure to nicorandil was substantially greater than that of the pro-drug Compound 8. Based on geometric mean metabolite to parent ratios, MW-corrected Cₘₐₓ values were between 51.0- to 103-fold higher than those of Compound 8 across the 40 mg and 80 mg doses. MW-corrected geometric mean AUC(0-tau) values were between 94.2 to 152-fold higher than those of Compound 8, based on the 40 mg and 80 mg doses. Results of the formal statistical analysis of dose-accumulation indicated accumulation was seen by Day 5 following the 40 mg dose, with GMRs of 1.82 for Cₘₐₓ and 2.37 for AUC(0-tau). No formal assessment of dose accumulation was performed following 80 mg dosing due to the low number of reliable results.

Pharmacokinetic and Statistical Conclusions for 1-cyclohexylethylamine

In part 2 of the study, following single dosing of Compound 8 Capsule at 40 mg and 80 mg, PK profiles for 1-cyclohexylethylamine were entirely BLQ for the 40 mg dose level for 7 out of the 8 subjects. Quantifiable concentrations were observed in all subjects at the 80 mg dose level. Following multiple oral dosing of either 40 mg or 80 mg Compound 8 for 5 days, quantifiable concentrations of 1-cyclohexylethaylamine were observed in all subjects. 1-cyclohexylethylamine appeared to form slowly with Tmax occurring between 6 hours and 12 hours post-dose over both dose levels and median Tₘₐₓ occurring at 10 hours and 8 hours post-dose on Day 1 and Day 5, respectively. Where calculable, the individual plasma half-life of 1-cyclohexylethylamine ranged from 13.5 hours to 31.4 hours over both dose levels. Exposure to 1-cyclohexylethylamine, based on geometric mean Cₘₐₓ and AUC(0-tau), increased between the 40 mg and 80 mg doses.

Clinical studies with nicorandil with 10 mg BID dosing show activity in contrast induced nephropathy. The pharmacokinetic parameters of nicorandil using 10 mg has a Cₘₐₓ of 107 (range of 44-148) ng/mL, AUC of 101 (range of 122-323) hour·ng/m, and a Tₘₐₓ of 0.43 (range 0.25-0.75) hours. While the clinical studies with nicorandil did not have the PK reported, one can extrapolate it from the healthy volunteer study of nicorandil. Tables C and D illustrate the therapeutic levels of 160 mg single dose and 80 mg BID dosing of Compound 8, which demonstrate the same efficacy as 10 mg dosing of nicorandil.

TABLE C
Compound 8 Phase I PK Data

| | Regimen | | | | |
|---|---|---|---|---|---|
| | A | B | C | D | E |
| | Dose Level | | | | |
| | 10 mg | 20 mg | 40 mg | 80 mg | 160 mg |
| | Status | | | | |
| | Fasted | Fastsed | Fasted | Fasted | Fasted |
| No. of Subjects | n = 6 | n = 6 | n = 6 | n = 5 | n = 5 |
| Tₘₐₓ (h) | NC | 4.00 | 4.00 | 4.017 | 4.00 |
| | | (3.05-4.02) | (3.00-6.05) | (3.00-4.18) | (3.00-6.17) |
| Cₘₐₓ (ng/mL) | NC | 7.73 (55.4) | 12.8 (71.0) | 47.4 (54.1) | 78.8 (21.4) |
| AUC(0-24) | NC | 20.0, 82.0 | 59.4 (11.0) | 146 (67.8) | 302 (52.8) |
| (ng · h/mL) | | (n = 2) | (n = 3) | | |
| T_{1/2} (h) | NC | NC | NC | NC | 1.77 (n = 1) |

TABLE D
Compound 8 Phase I PK Data

| | Regimen | | | |
|---|---|---|---|---|
| | F | F | G | G |
| | Dose Level | | | |
| | 80 mg BID | 80 mg BID | 40 mg BID | 40 mg BID |
| | Status | | | |
| | Day 1 | Day 5 | Day 1 | Day 5 |
| No. of Subjects | n = 7 | n = 7 | n = 7 | n = 7 |
| Tₘₐₓ (h) | 3.508 (3.00-4.00) | 4.017 (4.00-48.00) | 4.00 (3.00-4.00) | 4.00 (4.00-6.00) |
| | (n = 4) | (n = 3) | | |
| Cₘₐₓ (ng/mL) | 35.2 (41.6) | 74.3 (78.9) | 16.0 (47.2) | 29.3 (55.2) |
| | (n = 4) | (n = 3) | | |
| AUC(0-24) | 115 (60.9) | 345 (21.0) | 37.3 (61.0) | 108 (86.2) |
| (ng · h/mL) | (n = 3) | (n = 3) | (n = 7) | |
| T_{1/2} (h) | NA | NC | NC | NC |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

At least some of the chemical names of compounds as given and set forth in this application, may have been generated on an automated basis by use of a commercially available chemical naming software program, and have not been independently verified In the instance where the indicated chemical name and the depicted structure differ, the depicted structure will control. In the chemical structures where a chiral center exists in a structure but no specific stereochemistry is shown for the chiral center, both enantiomers associated with the chiral structure are encompassed by the structure.

1. A method for treating a disease or a condition related to kidney transplant, wherein the method comprises administering to the mammal an effective disease-treating or condition-treating amount of a compound according to formula (I): 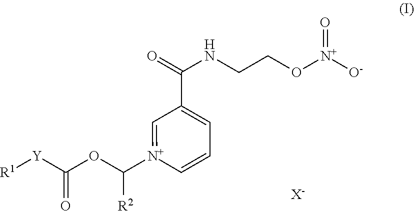
or a pharmaceutical composition thereof,
wherein X⁻ is a counter ion;
Y is —C(H)₂—, —O—, or —N(R₃)—;
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

2. (canceled)

3. The method of claim 1, wherein R² is H, Me, Et, or i-Pr.

4. The method of claim 1, wherein R² is H.

5. The method of claim 1, wherein Y is —C(H)₂—.

6. The method of claim 1, wherein Y is —O—.

7. The method of claim 1, wherein Y is —N(R³)—.

8. The method of claim 1, wherein the compound is according to formula (IIa), (IIb), or (IIc): 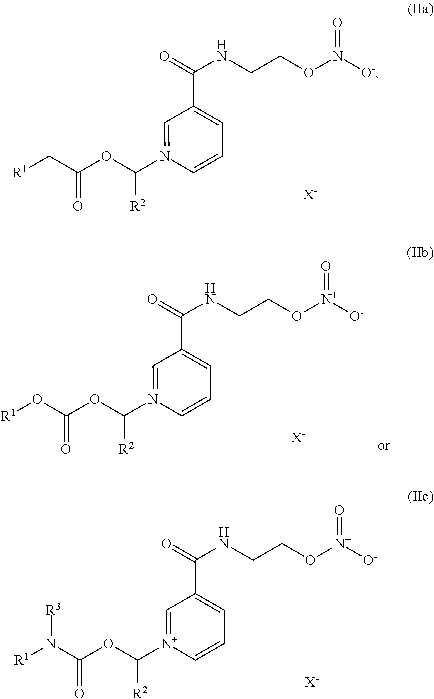
wherein
X⁻ is a counter ion;
R¹ is substituted or unsubstituted C₁-C₄ alkyl, substituted or unsubstituted C₃-C₇-cycloalkyl, or substituted or unsubstituted 4-7 membered heterocycloalkyl;
R² is H or C₁-C₄ alkyl; and
R³ is H or substituted or unsubstituted C₁-C₄ alkyl; or R¹ and R³ are joined together to form a 4-7 membered substituted or unsubstituted heterocycloalkyl.

9. The method of claim 1, wherein R¹ is substituted or unsubstituted C₁-C₄ alkyl.

10. The method of claim 1, wherein R¹ is C₁-C₄ alkyl, unsubstituted or substituted with C₃-C₇ cycloalkyl.

11. The method of claim 1, wherein R¹ is Me, Et, i-Pr, n-Pr, n-Bu, i-Bu, sec-Bu, or t-Bu.

12. The method of claim 1, wherein R¹ is Me or Et substituted with cyclopropyl, cyclobutyl, cyclopentyl or cyclohexyl.

13. The method of claim 1, wherein R¹ is C₃-C₇-cycloalkyl unsubstituted or substituted with Me or Et.

14. The method of claim 1, wherein R¹ is cyclopropyl, cyclobutyl, cyclopentyl or cyclohexyl.

15. The method of claim 1, wherein R³ is H or substituted or unsubstituted C₁-C₄ alkyl.

16. (canceled)

17. The method of claim 1, wherein R³ is Me, Et, i-Pr, n-Pr, n-Bu, i-Bu, sec-Bu, or t-Bu.

18. The method of claim 1, wherein R¹ and R³ are joined together to form a 4-7 membered substituted or unsubstituted heterocycloalkyl.

19. The method of claim 1, wherein R¹ and R³ are joined together to form pyrrolidinyl, piperidinyl, or morpholinyl.

20. The method of claim 1, wherein the compound is according to formula (IIIa), or (IIIb): 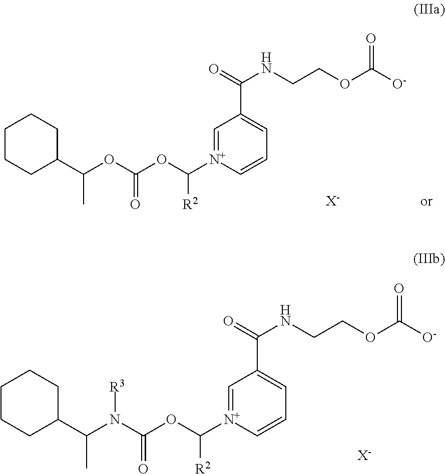
wherein
X⁻ is a counter ion; R² is H or C₁-C₄ alkyl; and
R³ is H or substituted or unsubstituted C₁-C₄ alkyl.

21. The method according to claim 20, wherein R³ is H, Me, Et, or i-Pr.

22. (canceled)

23. The method of claim 20, wherein R² is H or Me.

24. The method of claim 20, wherein the compound is according to formula (IIIb); and each R² and R³ is H.

25. The method of claim 1, wherein the compound is according to formula (IVa), or (IVb): 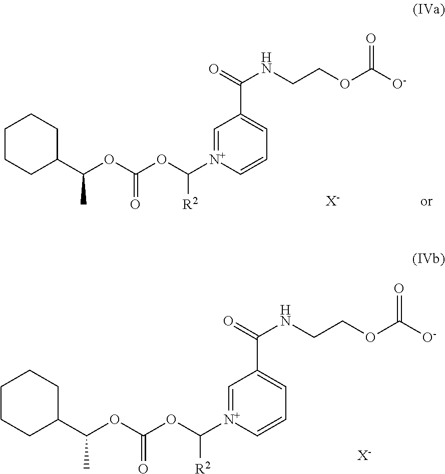
wherein X⁻ is a counter ion.

26. The method of claim 1,
wherein the compound is according to formula (Va), or (Vb): 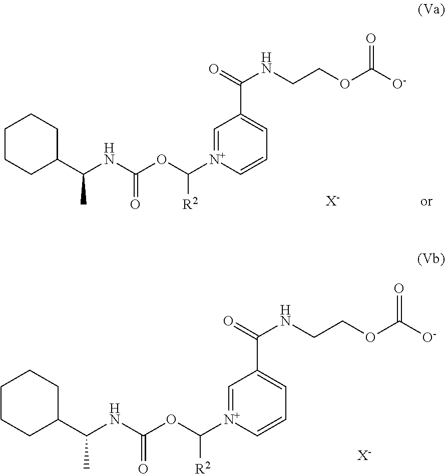
wherein X⁻ is a counter ion.

27. The method of claim 1, wherein X⁻ is at least one of iodide, chloride, fluoride, bromide, acetate, carbonate, chromate, citrate, fumarate, lactate, malonate, mesylate, nitrate, phosphate, tartrate, trifluoroacetate, succinate, sulphate, and sulphonate.

28. The method of claim 1, wherein X⁻ includes two counter ions independently selected from iodide, chloride, fluoride, bromide, acetate, carbonate, chromate, citrate, fumarate, lactate, malonate, mesylate, nitrate, phosphate, tartrate, trifluoroacetate, succinate, sulphate, and sulphonate.

29. The method of claim 1, wherein X⁻ is iodide.

30. A method for treating a disease or a condition related to kidney transplant, wherein the method comprises administering to the mammal an effective disease-treating or condition-treating amount of nicorandil, or a derivative or prodrug thereof, or a pharmaceutically acceptable salt thereof.

31. The method of claim 1, wherein the compound is in a pharmaceutical composition.

32. (canceled)

33. (canceled)

34. The method of claim 1, wherein the disease or condition is selected from Transplant related Ischemia, Acute Kidney Injury (AKI) post kidney transplant, Delayed Graft Function (DGF), Primary Non Function (PNF), Prolonged Graft Ischemia, Primary Graft Failure, Rejection with Chronic Graft Failure, Ischemia Reperfusion (IR), Post-ischemic Acute Tubular Necrosis (ATN), Chronic Kidney Disease, Acute Kidney Failure, and/or Acute Kidney Disease, and combinations thereof.

35. The method of claim 1, wherein the disease or condition is Delayed Graft Function, Prolonged Graft Ischemia, Ischemia Reperfusion injury, Post-ischemic Acute Tubular Necrosis, Rejection with Chronic Graft Failure, or combinations thereof.

36. (canceled)

37. (canceled)

38. (canceled)

39. (canceled)

40. The method of claim 1, wherein the method comprises administering daily to the mammal an effective disease-treating or condition-treating amount of a compound according to formula (I) starting before the time of a kidney transplant, starting at the time of the kidney transplant, or staring after the time of the kidney transplant.

41. (canceled)

42. (canceled)

43. The method of claim 40, wherein the administering daily to the mammal an effective disease-treating or condition-treating amount of a compound according to formula (I) has a duration of 1 day to 5 days.

44. The method of claim 40, wherein the administering daily to the mammal an effective disease-treating or condition-treating amount of a compound according to formula (I) is in an amount of 80 mg to 160 mg, or wherein the administering daily to the mammal an effective disease-treating or condition-treating amount of a compound according to formula (I) is in an amount of 20 mg BID to 80 mg BID.

45. (canceled)

46. The method of claim 40, wherein the administering daily to the mammal an effective disease-treating or condition-treating amount of a compound according to formula (I) is in an amount of 20 mg BID, 40 mg BID, or 80 mg BID.
